SEQUENCE LISTING (7 sequences)

SEQ ID NO: 1 - DNA, 6277 bp, Homo sapiens
aaaaaaaaaaaaaaaaaaagaaaaaaaaccccgccggatccgaccgccactttcaaaaccccccaccgctctagaaccgcgggagcttccgtccctgagtagaattcgagggtgtaaagaagaggaaggggaaaaatatcttgtaccagcccaggggtgaagaagcccccggcctgagaaagaaggaggagtgggggaggcgaacagtctcgttgctgcctctgtgtacgctgaggggggaggtggccaccgagtactaaattcacttgggaataaaagaaaaacataagaaaattataagagaaaggaattgtcttagaagaaagaaggcaagccaccattttacccacgtaaatatatgaatatatttctgacattgaggtgttccagaagatgataaagaaatgatagcagctccagaaataccaactgattttaatctactacaggagtcagaaacacatttttcttctgacacagattttgaagatatcgaaggaaaaaaccaaaagcaaggcaaaggcaaaacttgtaaaaaaggcaaaaagggcccagcagaaaagggcaaaggtggaaatggaggaggaaaacctccttctggtccaaaccgaatgaatggtcatcaccaacagaatggagtggaaaacatgatgttgtttgaagttgttaaaatgggcaagagtgctatgcagtcggtggtagatgattggatagaatcatacaagcatgaccgagatatagcacttcttgaccttatcaacttttttattcagtgttcaggctgtaaaggagttgtcacagcagaaatgtttagacatatgcagaactctgagataattcgaaaaatgactgaagaattcgatgaggatagtggagattatccacttaccatggctggtcctcagtggaagaagttcaaatccagtttttgtgaattcattggcgtgttagtacggcaatgtcaatatagtatcatatatgatgagtatatgatggatacagtcatttcacttcttacaggattgtctgactcacaagtcagagcatttcgacatacaagcaccctggcagctatgaagttgatgacagctttggtgaatgtggcactaaatcttagcattaatatggataatacacaaagacaatatgaagcagaacggaataaaatgattggaaaacgagccaatgagaggctagaactcctgctacaaaagcggaaagagcttcaggaaaatcaagatgaaatagaaaatatgatgaatgcaatatttaaaggagtgtttgtacatagataccgtgatgcgatagctgaaattcgagctatttgcattgaagagattggcatttggatgaagatgtatagtgatgcctttcttaatgacagttatttaaaatatgttggttggactatgcatgataagcaaggtgaagtaagactcaaatgtcttactgctctacaagggctttattataacaaagagcttaattccaaactggaactttttaccagtcggttcaaggatagaattgtgtctatgacccttgacaaagaatatgatgttgcagtacaagcaataaaattactcactcttgttttacagagtagtgaagaagttctcactgcagaagattgtgaaaatgtctatcatctggtttattcagctcaccggccagtagcagtagcagctggagaatttctctacaaaaagctcttcagtcgtagagatccagaggaggatggaatgatgaaaagaagaggaagacaaggtccaaatgccaaccttgttaagacattggtttttttctttctagaaagtgagttacatgagcatgcagcataccttgtggatagcatgtgggactgtgctactgagctgctgaaagactgggaatgtatgaatagcttgttactggaagagccacttagtggagaggaagcactaacagataggcaagagagtgctctgattgaaataatgctttgtaccattagacaagcggctgaatgtcatcctcccgtgggaagagggacaggaaaaagggtgcttacagcaaaggagaagaagacacagttggatgataggacaaaaatcactgagctttttgccgtggcccttcctcagttattagcaaaatactctgtagatgcagaaaaggtgactaacttgttgcagttgcctcagtactttgatttggaaatatataccactggacgattagaaaagcatttggatgccttattgcgacagatccggaatattgtagagaagcacacagatacagatgttttggaagcatgttctaaaacttaccatgcactctgtaatgaagagttcacaatcttcaacagagtagatatttcaagaagtcaactgatagatgaattggcagataaatttaaccggcttcttgaagattttctgcaagagggtgaagaacctgatgaagatgatgcatatcaggtattgtcaacattgaagaggatcactgcttttcataatgcccatgacctttcaaagtgggatttatttgcttgtaattacaaactcttgaaaactggaatcgaaaatggagacatgcctgagcagattgttattcacgcactgcagtgtactcactatgtaatcctttggcaacttgctaagataactgaaagcagctctacaaaggaggacttgctgcgtttaaagaaacaaatgagagtattttgtcagatatgtcaacattacctgaccaacgtgaatactactgttaaggaacaggccttcactattctgtgtgatattttgatgatcttcagccatcagattatgtcaggagggcgtgacatgttagagccattagtgtatacccctgattcttcattgcagtctgagttgctcagctttattttggatcatgtcttcattgaacaggatgatgataataatagtgcagatggtcagcaagaggatgaagccagtaaaattgaagctctgcacaagagaagaaatttacttgcagcattttgtaagctaattgtatatactgtggtggagatgaatacagctgcagatatcttcaaacagtatatgaagtattataatgactatggagatatcatcaaagaaacaatgagtaaaacaaggcagatagacaaaattcagtgtgctaagacccttattctcagtctgcaacagctttttaatgaaatgatacaagaaaatggctataattttgatagatcatcctctacatttagtggcataaaagaacttgctcgacgttttgctttaacttttggacttgatcagttgaaaacaagagaagccattgccatgctacacaaagatggcatagaatttgcttttaaagagcctaatccgcaaggggagagccatccacctttaaatttggcatttcttgatattctgagtgaattttcttctaaactacttcgacaagacaaaagaacagtgtatgtttacttggaaaagttcatgacctttcagatgtcactccgaagagaggatgtgtggcttccactgatgtcttaccgaaattctttgctagctggtggtgatgatgacaccatgtcagtcattagtggaatcagcagccgggggtcaacagtacggagtaaaaaatcaaaaccatctacaggaaaacggaaagtggttgagggcatgcagctttcactcactgaagaaagtagtagtagtgacagtatgtggttaagcagagaacaaacactgcacacccctgttatgatgcagacaccacaactcacctccactattatgagagagcccaaaagattacggcctgaggatagcttcatgagtgtttatccaatgcagactgaacatcatcaaacacctcttgattataacacgcaggtaacatggatgttagctcaaagacaacaagaggaagcaaggcaacagcaggagagagcagcaatgagctatgttaaactgcgaactaatcttcagcatgccattcggcgtggcacaagcctaatggaagatgatgaagagccaattgtggaagatgttatgatgtcctcagaagggaggattgaggatcttaatgagggaatggattttgacaccatggatatagatttgccaccatcaaagaacagacgagagagaacagaactgaagcctgatttctttgatccagcttcaattatggatgaatcagttcttggagtgtcaatgttttaataccagtacacaattaaatctgtggtgaagtcattttctaagtggaagaggaaattttaaagtgtggtagatacagtgaaattctgtacagatttttctctaaggagaatatgacatgcttatgcttaccaagatcaagtgcattgaggggcagttttgtttgcctgaataaacgtaaaggacaagtaaacaatttgatgataagctacagtttttcttagaaagtaaatattttatttatgcgctgttagttggcttttgaatcgattatttcatgcttttttttaaaaaaaaaaaaaaacaaaataacaatctgaagaggcatttggtacagatatgaattctcttacatttatttactggttgtactaaataatgatgacctctgctggatttctgtttacatccagaaaacaatgttaaggatgtatttattcccctaccctgaagaaagtgtaggatagaattgtttttagcattctaaatttaaatgcttaaaacgtcaatcaacaaaactttgttttaaatattgtaattgtggagaaaagtaaacttataagcagaacttttacaattttttcatctaaaagtattttaagatatttttaaaatccaagagcttctctatacttttcagaaatatccagatgcagtgaactgccagaaggtaaccagtctcaaacatgcttatcccattatcaaccctgaaagtttgcttgtcctttaagataaaaatgtaatgttgtgatattccttccagtaatgccactgtattttgtctccaaataaaagaagcttattgtagtatgtttgcagaaaaattctaaacaaaaattatacagcttattagagtgtgggaatagggatctaaattttaaataaaattatatatatatataaattggtgctgattttataattgcgcagtttgtttagttttttcttacttttaaattccaacttaaaattatgaggtttcagaaatatattgaaagtttaacaatgtttaaaaatagaaaagcatgagtgttcatgctttaaaatgatttttaaatttgtattttatattgttttatctatctgtctttgcaagcagtcttcaggttaaagatacttctaacaggttacagtacatttcctctgtatgtaaattagatgggataatagaattcataacccataatattctttgaaagctaagctttaaacttcattttatgtcctttcacaaataaattagtttaaaacagaaagtggctacttgccattttgacatcaactcattttgcgaggcttaggcagctagacatcgtttaaaacaaaatattaacttatattacatgtgtatctatctattgtcagtcgtctctcagttcttgaggtatattattttaatcattccatgccttaatatgcttgcaatacaagaatatcttcagatgggtgaataccaaaaggctttcagtttttagtcagaaatcaagcattgggctgtggtagccaaaaaccataggttagctaaaaagatcatgatacaattattttattaagtcatggttaataacaaatgaatccagacttgtctaacagattttccatcaacaaatattgttatgtgcaaaagtattgcctatgttgttttacacaccactgcattaactagaactgctgagaggactgtatatatgattttaaacctaagttgattttttttctcactcttgaaaggagtacttctttgtgaaagcagttcttacagctttgttttcaaccagctaaaaatgttttatatattactctaacctgttgtcctccacattctattgtcctaattgtactgttttctgatttgtatttatgtcttgagacagtaactttttgaataaaaataaacctacagtatgttgtatgttttctcttgtactcaaagggggagggtggctataaatggtttgcaaatttatatctattatcacatcttttaatgtgtttggggaataatttatagagaataccatcagtttatatttttaataaatcatatgtatttacaatgaaaaaaaaaa

SEQ ID NO: 2 - DNA, 30 bp, Homo sapiens
tacttttactctttaaaaatagctatgaag

SEQ ID NO: 3 - DNA, 20 bp, Homo sapiens
tttactctttaaaaatagct

SEQ ID NO: 4 - DNA, 25 bp, Homo sapiens
tactgctctacaagggctttattat

SEQ ID NO: 5 - DNA, 30 bp, Homo sapiens
ccccattcagtgatgcgatagctgaaattc

SEQ ID NO: 6 - protein, 1268 aa, Homo sapiens
MIAAPEIPTDFNLLQESETHFSSDTDFEDIEGKNQKQGKGKTCKKGKKGPAEKGKGGNGGGKPPSGPNRMNGHHQQNGVENMMLFEVVKMGKSAMQSVVDDWIESYKHDRDIALLDLINFFIQCSGCKGVVTAEMFRHMQNSEIIRKMTEEFDEDSGDYPLTMAGPQWKKFKSSFCEFIGVLVRQCQYSIIYDEYMMDTVISLLTGLSDSQVRAFRHTSTLAAMKLMTALVNVALNLSINMDNTQRQYEAERNKMIGKRANERLELLLQKRKELQENQDEIENMMNAIFKGVFVHRYRDAIAEIRAICIEEIGIWMKMYSDAFLNDSYLKYVGWTMHDKQGEVRLKCLTALQGLYYNKELNSKLELFTSRFKDRIVSMTLDKEYDVAVQAIKLLTLVLQSSEEVLTAEDCENVYHLVYSAHRPVAVAAGEFLYKKLFSRRDPEEDGMMKRRGRQGPNANLVKTLVFFFLESELHEHAAYLVDSMWDCATELLKDWECMNSLLLEEPLSGEEALTDRQESALIEIMLCTIRQAAECHPPVGRGTGKRVLTAKEKKTQLDDRTKITELFAVALPQLLAKYSVDAEKVTNLLQLPQYFDLEIYTTGRLEKHLDALLRQIRNIVEKHTDTDVLEACSKTYHALCNEEFTIFNRVDISRSQLIDELADKFNRLLEDFLQEGEEPDEDDAYQVLSTLKRITAFHNAHDLSKWDLFACNYKLLKTGIENGDMPEQIVIHALQCTHYVILWQLAKITESSSTKEDLLRLKKQMRVFCQICQHYLTNVNTTVKEQAFTILCDILMIFSHQIMSGGRDMLEPLVYTPDSSLQSELLSFILDHVFIEQDDDNNSADGQQEDEASKIEALHKRRNLLAAFCKLIVYTVVEMNTAADIFKQYMKYYNDYGDIIKETMSKTRQIDKIQCAKTLILSLQQLFNEMIQENGYNFDRSSSTFSGIKELARRFALTFGLDQLKTREAIAMLHKDGIEFAFKEPNPQGESHPPLNLAFLDILSEFSSKLLRQDKRTVYVYLEKFMTFQMSLRREDVWLPLMSYRNSLLAGGDDDTMSVISGISSRGSTVRSKKSKPSTGKRKVVEGMQLSLTEESSSSDSMWLSREQTLHTPVMMQTPQLTSTIMREPKRLRPEDSFMSVYPMQTEHHQTPLDYNTQVTWMLAQRQQEEARQQQERAAMSYVKLRTNLQHAIRRGTSLMEDDEEPIVEDVMMSSEGRIEDLNEGMDFDTMDIDLPPSKNRRERTELKPDFFDPASIMDESVLGVSMF

SEQ ID NO: 7 - DNA, 19 bp, Homo sapiens
ttatagtattataatgact

(19) United States
(12) Patent Application Publication
(10) Pub. No.: US 2013/0029865 A1
(43) Pub. Date: Jan. 31, 2013

(54) Stromal Antigen 2 (STAG2) Compositions and Methods

(71) Applicants: David SOLOMON, Washington, DC (US); Jung-Sik Kim, Clarksburg, MD (US); Todd Waldman, Bethesda, MD (US)

(72) Inventors: David SOLOMON, Washington, DC (US); Jung-Sik Kim, Clarksburg, MD (US); Todd Waldman, Bethesda, MD (US)

(73) Assignee: Georgetown University, Washington, DC (US)

(21) Appl. No.: 13/547,966

(22) Filed: Jul. 12, 2012

Related U.S. Application Data
(60) Provisional application No. 61/508,437, filed on Jul. 15, 2011.

(51) Int. Cl. C12N 15/12; G01N 33/53; C12Q 1/68; C07K 14/47; C40B 30/04

(52) U.S. Cl. 506/9; 536/23.5; 530/350; 435/6.1; 435/7.1

(57) ABSTRACT

Compositions and methods related to stromal antigen 2 (STAG2) and its role in diverse human cancers, including nucleic acids, polypeptides, vectors, cells and cell lines.

### CROSS-REFERENCE TO RELATED APPLICATIONS

Priority is claimed to U.S. Provisional Patent Application No. 61/508,437, filed Jul. 15, 2011, which is incorporated by reference herein in its entirety.

### STATEMENT REGARDING FEDERALLY SPONSORED RESEARCH OR DEVELOPMENT

This invention was made with government support under Grants R01CA115699 and R21CA143282 awarded by the National Institutes of Health. The government has certain rights in inventions disclosed herein.

### TECHNICAL FIELD OF THE INVENTION

The invention relates generally to compositions and methods concerning stromal antigen 2 (STAG2) nucleic acids and polypeptides with roles in diverse human cancers, particularly bladder cancer, glioblastoma multiforme, Ewing's sarcoma and melanoma. The aforementioned compositions and methods also concern related vectors, cells and cell-lines.

### BACKGROUND OF THE INVENTION

One hallmark of cancer is chromosomal instability, resulting in aneuploidy, translocations, loss of heterozygosity, and other chromosomal aberrations. This instability is an early event in cancer pathogenesis and is thought to be required for generating the large number of genetic lesions required for a cell to undergo malignant transformation.

It has long been thought that mutational inactivation of genes that control chromosomal segregation is responsible for aneuploidy in human cancer. Targeted overexpression or genetic inactivation of factors involved in chromatin condensation, mitotic checkpoint, and chromosome segregation has demonstrated that these genes can function to maintain chromosomal stability.

However, analysis of human cancer samples has yielded few examples of putative instability genes that are mutated or deleted at an appreciable frequency, and in no case has chromosomal instability in human cancer been reverted by correction of a naturally occurring gene mutation. Accordingly, solutions to identifying sources of chromosomal instability in cancer cells are needed so that agents that selectively target these sources and/or cancer cells resulting therefrom may be identified and/or developed.

### BRIEF SUMMARY OF THE INVENTION

The present invention relates to STAG2 polynucleotides, polypeptides, vectors and cells expressing STAG2 polynucleotides that are associated with a variety of cancers, particularly human cancers, and more particularly human cancers resulting from chromosomal instability and chromosomal aberrations such as aneuploidy. STAG2 encodes a 141 kDa protein that is a core component of the cohesin complex, a multimeric protein complex with ring-like structure that is required for cohesion of sister chromatids following DNA replication and is cleaved at the metaphase to anaphase transition to enable chromosome segregation. The polynucleotides, polypeptides, vectors and cells of the present invention may be used to, inter alia, detect STAG2-related abnormalities in patient samples and screen candidate compounds that may selectively kill or otherwise inhibit the growth of cancer cells that are either STAG2-deficient or express mutated forms of STAG2.

In particular embodiments, the invention relates to isolated polynucleotides encoding a STAG2 polypeptide associated with at least one chromosomal aberration (e.g., aneuploidy).

In other embodiments, the invention relates to isolated polynucleotides capable of detecting a STAG2 polynucleotide associated with at least one chromosomal aberration by specifically hybridizing to the STAG2 polynucleotide or its complement under specified hybridization and wash conditions.

In some embodiments, the invention relates to STAG2 knock-in cells and cell-lines (e.g., H4 STAG2 KI post-Cre 8-1 cells, 42MGBA STAG2 KI cells) and STAG2 knock-out cells and cell-lines (e.g., HCT116 STAG2 KO 7 cells), as well as vectors for creating the same and methods of using those vectors.

In other embodiments, the invention relates to methods of determining the presence of a STAG2 polynucleotide or polypeptide associated with at least one chromosomal aberration comprising: a) obtaining a biological sample from a subject (e.g., human patient) comprising at least one cell having at least one chromosomal aberration; and b) detecting the presence of a STAG2 polynucleotide or polypeptide associated with the at least one chromosomal aberration. In some of these embodiments, the STAG2 polynucleotide comprises at least one nucleotide insertion, nucleotide deletion, missense mutation, or nonsense mutation in the STAG2 gene that may be identified by using one or more STAG2 reference sequences. In some embodiments, a deletion may comprise large sections of a STAG2 gene, including the entire gene.

In other embodiments, the invention relates to methods of determining whether a subject is at risk for developing cancer comprising a) obtaining a biological sample from a subject comprising at least one cell; and b) detecting the presence or absence of a STAG2 polynucleotide or polypeptide in the at least one cell, wherein the presence or absence of the STAG2 polynucleotide or polypeptide is correlated with a risk for cancer. In some of these embodiments, the STAG2 polynucleotide comprises at least one nucleotide insertion, nucleotide deletion, missense mutation, or nonsense mutation in the STAG2 gene that may be identified by using one or more STAG2 reference sequences. In some embodiments, a deletion may comprise large sections of a STAG2 gene, including the entire gene.

In other embodiments, the invention relates to methods of A method of determining a clinical course of treatment for a subject with at least one tumor comprising: (a) in a sample of the tumor obtained from the subject, detecting the presence or absence of a STAG2 polynucleotide or polypeptide associated with at least one chromosomal aberration in a tumor sample obtained from the subject; (b) correlating the presence or absence of the STAG2 polynucleotide or polypeptide with the clinical course of treatment. Many tumors have reduced or completely undetectable levels of STAG2, which are due to a wide variety of mutations in STAG2, many of which are described in Table 1.

In other embodiments, the invention relates to methods of identifying an agent that affects the viability of a cell comprising a STAG2 polynucleotide or polypeptide associated with at least one chromosomal aberration (e.g., aneuploidy) comprising a) administering the agent to a sample comprising at least one cell comprising a STAG2 polynucleotide or polypeptide associated with at least one chromosomal aberration; and b) determining whether the agent affects the viability of the at least one cell. In particular embodiments, the at least one cell is a homozygous STAG-2 deficient cell (e.g., an H4 cell, 42MGBA, HCT116 STAG2 KO 7 cell).

In other embodiments, the invention relates to methods of identifying an agent that selectively affects a cell comprising a STAG2 polynucleotide or polypeptide associated with at least one chromosomal aberration (e.g., aneuploidy) comprising a) administering the agent to a first sample comprising at least one cell comprising a STAG2 polynucleotide or polypeptide associated with at least one chromosomal aberration; b) administering the agent to a second sample comprising at least one cell comprising at least one cell comprising a STAG2 polynucleotide or polypeptide that is not associated with at least one chromosomal aberration and is otherwise isogenic with the at least one cell of the first sample; and c) determining whether the agent selectively affects the at least one cell comprising a STAG2 polynucleotide or polypeptide associated with at least one chromosomal aberration.

### BRIEF SUMMARY OF THE SEVERAL VIEWS OF THE DRAWINGS

FIG. 1: Frequent genetic lesions of the cohesin complex subunit STAG2 in diverse human cancers. (A) Identification of a homozygous deletion of the STAG2 gene in the glioblastoma cell line U138MG. Copy number plots along the X chromosome for normal human astrocytes (NHAs) derived from a female patient and a male patient, A172 glioblastoma cells from a male patient, U87MG glioblastoma cells from a male patient, and U138MG glioblastoma cells from a male patient are shown. A genomic deletion between 122.930-123.226 Mb is present in U138MG cells that encompasses the STAG2 gene located at 123.094-123.235 Mb. (B-G) Western blots for STAG2 and α-tubulin demonstrating complete loss of STAG2 expression in 3/21 glioblastoma, 5/9 Ewing's sarcoma, 1/10 melanoma, 2/20 hematologic cancer, 1/6 cervical cancer, and 1/4 kidney cancer cell lines. (H) Diagram of the STAG2 protein depicting the location of identified mutations. Diamonds indicate missense mutations. Squares indicate nonsense, frameshift, and splice site truncating mutations. Bar indicates intragenic deletion in SR lymphoma cells. Stromal antigen (STAG) domain, amino acids 154-273. Stromalin conservative domain (SCD), amino acids 293-378. Hashes indicate known serine/threonine phosphorylation sites.

FIG. 2: Single hit genetic inactivation causes loss of STAG2 in diverse human tumor types. (A) STAG2 sequence traces from genomic DNA and RNA prepared from STAG2-32 Ewing's sarcoma cells, derived from a female patient. Whereas the genomic DNA sequence is heterozygous for a single nucleotide insertion, the mRNA sequence is derived exclusively from the mutant allele on the active X chromosome. (B) STAG2 immunohistochemistry was performed on tissue microarrays composed of glioblastoma, melanoma, lymphoma, and Ewing's sarcoma primary tumors. Simultaneous α-tubulin staining on a consecutive section of the microarrays was used as a positive control to confirm that STAG2-negative cores were accessible for staining by an unrelated antibody. The depicted STAG2-deficient lymphoma demonstrates intratumoral heterogeneity. (C) The number of tumors successfully assessed by immunohistochemistry and the fraction demonstrating complete loss of STAG2 expression is shown.

FIG. 3: Targeted correction of the endogenous mutant allele of STAG2 in human glioblastoma cells restores sister chromatid cohesion. (A) Diagram depicting the targeted homologous recombination event for correcting the endogenous STAG2 nonsense mutation at codon 653 in 42MGBA cells. In the initial step, an AAV-based targeting vector was created for the purpose of correcting the exon 20 mutation, leaving behind a FLOXed splice acceptor-IRES-Neo^{R} gene in the subsequent intron. Clones with targeted integration were identified by PCR and DNA sequencing (FIG. 19). These cells (pre-Cre clones) were then transiently infected with a Cre-expressing adenovirus, and completed STAG2 knock-in (KI) clones in which the splice acceptor-IRES-Neo^{R} gene had been deleted by Cre/LoxP recombination were identified by screening for G418-sensitivity (post-Cre clones). Black triangles indicate LoxP sites. (B) Western blot demonstrating STAG2 re-expression in 42MGBA STAG2 KI clones. 42MGBA parental cells and two non-recombinant clones in which the STAG2 KI vector randomly integrated fail to express STAG2 protein. Two pre-Cre clones in which the STAG2 KI vector had integrated by homologous recombination but in which the splice acceptor-IRES-Neo^{R} had not yet been removed similarly fail to express STAG2 protein because the STAG2 transcript gets spliced to the IRES-Neo^{R} gene rather than to STAG2 exon 21. Three STAG2 KI post-Cre clones in which IRES-Neo^{R} was successfully removed via Cre/LoxP recombination express physiologic levels of corrected STAG2 protein, comparable to the levels in 8MGBA and U87MG glioblastoma cells with unmodified wild-type STAG2 alleles. (C) Representative micrographs showing examples of chromosome spreads from mitosis-arrested STAG2-deficient H4 cells with cohered, parallel, and fully separated sister chromatids. Arrows indicate each sister chromatid in a mitotic chromosome. Arrowhead points to the centromere. (D) Quantification of sister chromatid cohesion defects in STAG2-deficient cells. Isogenic sets of STAG2-proficient and deficient cells were arrested in mitosis using taxol or nocodazole, Giemsa stained, and assayed for chromosome cohesion.

FIG. 4: Correction of mutant STAG2 alleles in human glioblastoma cells does not globally alter gene expression profile but reduces chromosomal instability. (A) Affymetrix GeneChip Human Gene 1.0 ST arrays were used to generate gene expression profiles in parental 42MGBA cells, two independently derived 42MGBA STAG2 KI pre-Cre clones, and three independently derived 42MGBA STAG2 KI post-Cre clones. Composite expression profiles of the three STAG2-mutant cells were plotted against the composite expression profiles of the three STAG2-corrected cells. The 16 genes whose expression was modulated >1.5-fold are displayed in table S4. (B) Asynchronously proliferating STAG2-deficient and proficient H4 cells were fixed in ethanol, stained with propidium iodide, and analyzed by flow cytometry. Representative histograms are shown with the DNA content of 17,600 cells plotted for both clones. The width of the 2N and 4N peaks is substantially greater in STAG2-deficient than in STAG2-corrected H4 cells. (C) Quantification of the coefficient of variance (a measure of variation in DNA content within a cell population) of the 2N peak from asynchronously proliferating STAG2-deficient and proficient H4 and HCT116 cells is shown. *, p<0.05. (D-E) Isogenic STAG2-proficient and deficient cells were arrested in metaphase and karyotypes prepared using Wright's Stain. Chromosomes were counted in 100 cells for each cell line to determine the diversity of chromosome counts within the cell population. Chromosome counts are shown in FIG. 23, and distribution curves from these data are shown here for STAG2-proficient and deficient H4 cells (D) and HCT116 cells (E).

FIG. 5: PCR confirmation of STAG2 genomic deletion in U138MG glioblastoma cells identified by copy number microarray. PCR for multiple STAG2 exons using genomic DNA from U138MG cells as template resulted in no amplification products, whereas PCR using genomic DNA from A172 and U87MG glioblastoma cells with intact STAG2 loci yielded amplification products at the expected molecular weight. PCR for the WTX gene that resides 60 Mb centromeric to the STAG2 gene on the X chromosome yielded amplification products at the predicted molecular weight in all three glioblastoma cell lines.

FIG. 6: STAG2 mutations identified in glioblastoma cells. (A) H4 cells have a 25 bp insertion/duplication in exon 12 resulting in a frameshift and early truncation of the encoded STAG2 protein. (B) 42MGBA cells have a nonsense mutation in codon 653 in exon 20 resulting in early truncation of the encoded STAG2 protein.

FIG. 7: STAG2 mutations identified in glioblastoma tumor samples. (A) GBM p785 has a somatic missense mutation at codon 299 in exon 11, resulting in an aspartic acid to alanine change in the stromalin conservative domain (SCD) of STAG2. (B) GBM 14 has a G>C mutation in the canonical splice acceptor of exon 9. (C) GBM 44 has a two by deletion (AA) in exon 9 causing a frameshift and early truncation of the encoded STAG2 protein. (D) SF7300 has a C>T mutation in the splice acceptor of exon 11.

FIG. 8: Human cancer cell lines in which no loss of STAG2 expression was observed. (A-I) Western blots for STAG2 and α-tubulin on protein isolated from 6 neuroblastoma, 14 lung, 13 colorectal, 3 gastric, 5 pancreatic, 7 gynecologic, 6 prostate, 5 bone, and 27 breast cancer cell lines.

FIG. 9: Homozygous genomic deletion of STAG2 in ES-8 Ewing's sarcoma and LOX IMVI melanoma cells. (A) PCR for exon 1 in the 5′ untranslated region of the STAG2 gene using ES-8 genomic DNA as template resulted in no amplification product at the correct molecular weight. PCR for all coding exons of STAG2 using genomic DNA from LOX IMVI cells as template yielded no amplification products at the correct molecular weight. PCR using genomic DNA from A172 and U87MG cells with intact STAG2 loci yielded amplification products at the expected molecular weight for each STAG2 exon. (B) Subsequent analysis of Affymetrix SNP 6.0 copy number array data for ES-8 and LOX IMVI cells revealed focal homozygous deletions encompassing the STAG2 locus.

FIG. 10: Intragenic deletion of STAG2 coding exons 28 to 30 in SR immunoblastic lymphoma cells in which no STAG2 protein was detected. (A) PCR for STAG2 coding exons 28 to 30 on genomic DNA from SR cells resulted in no amplification products, whereas PCR on genomic DNA from A172 and U87MG cells with intact STAG2 loci yielded amplification products at the expected molecular weight. PCR for STAG2 coding exons 27 and 31 yielded amplification products at the predicted molecular weight in all three cell lines. (B) RT-PCR amplification of STAG2 from total RNA isolated from SR cells revealed the absence of sequence from the deleted coding exons 28 to 30 and the inappropriate junction of exon 27 with exon 31 in the maturely spliced STAG2 mRNA.

FIG. 11: STAG2 mutations identified in Ewing's sarcoma and cervical carcinoma cells. (A) SK-ES-1 cells have a nonsense mutation in codon 735 in exon 23 resulting in early truncation of the encoded STAG2 protein. (B) STAG2-252 cells have a one by insertion (T) in exon 20 causing a frameshift and early truncation of the encoded STAG2 protein. (C) A4573 cells have a heterozygous one by deletion (A) in exon 25 causing a frameshift and early truncation of the encoded STAG2 protein. Sequencing of the STAG2 mRNA from A4573 cells demonstrated that only the mutant allele is expressed. (D) CaSki cells have a 20 bp deletion that includes the intron, splice acceptor, and coding sequence of exon 9 causing early truncation of the encoded STAG2 protein.

FIG. 12: Mutation traces of malignant melanoma and Ewing's sarcoma tumor samples with STAG2 mutations. (A) MM 29T has a somatic 6 bp duplication, causing an insertion of Asp-Met at codon 225 in the stromal antigen (STAG) domain of the encoded STAG2 protein. (B) ES 37 has a somatic A>G mutation 8 bp upstream of the initiating methionine in the putative Kozak consensus sequence of STAG2.

FIG. 13: Loss of STAG2 expression in cancer cells with heterozygous mutations of STAG2 is not reversible by inhibition of DNA methylation. STAG2-71 cells (derived from a male patient, harboring wild-type STAG2), STAG2-32 cells (female patient, heterozygous frameshift mutation of STAG2), A4573 cells (female patient, heterozygous frameshift mutation of STAG2), and SK-ES-1 cells (male patient, homozygous nonsense mutation of STAG2) were cultured in the presence or absence of 10 μM 5-aza-2-deoxycytidine for 96 hours. Total protein was harvested in RIPA buffer and assayed for STAG2 expression by Western blot.

FIG. 14: Validation of a STAG2 antibody for detection of STAG2 expression by immunohistochemistry in formalin-fixed, paraffin-embedded tissue. (A) STAG2-proficient (H4 non-recombinant clone 10) and STAG2-deficient (H4 STAG2 KI post-Cre clone 8-1) cells were fixed in 4% formalin, immersed in Histogel, embedded in paraffin, and then sectioned onto the same slide for simultaneous staining Immunohistochemistry was performed using the STAG2 clone J-12 mouse monoclonal antibody (Santa Cruz Biotechnology, sc-81852), which binds to an epitope near the C-terminus of the protein that is absent in cells with truncating mutations of the STAG2 gene (e.g., H4 cells). Antibody complexes were visualized by 3,3′-diaminobenzidine enzymatic reaction, and counterstaining with hematoxylin was performed. STAG2 staining was completely absent in non-recombinant H4 cells but was intensely present in the STAG2-corrected H4 clone. This staining was observed exclusively in the nucleus of each cell without significant cell-to-cell variation in expression level, consistent with previous reports on the localization and expression of STAG2. (B) Similar absence of STAG2 staining by immunohistochemistry was observed in additional cell lines harboring truncating mutations of STAG2, including 42MGBA cells shown here.

FIG. 15: Expression of STAG2 in non-neoplastic tissues. Immunohistochemistry with STAG2 and α-tubulin antibodies to normal non-neoplastic tissue is shown from appendix, lymph node, skeletal muscle, thymus gland, and skin. Robust expression of STAG2 was ubiquitously observed in all non-neoplastic tissues studied.

FIG. 16: STAG2 immunohistochemistry of additional glioblastoma tumors.

FIG. 17: STAG2 immunohistochemistry of additional melanoma tumors.

FIG. 18: STAG2 immunohistochemistry of additional Ewing's sarcoma tumors.

FIG. 19: PCR screen and DNA sequence confirmation of 42MGBA STAG2 knock-in clones. (A) 42MGBA cells were infected with an AAV-STAG2 KI vector as depicted in FIG. 3(A). Individual G418-resistant clones were established by limiting dilution in 96-well plates, genomic DNA prepared, and tested by PCR for homologous integration of the targeting vector. Clones with random integration of the targeting vector generate a single 1.3 kb band, whereas clones with targeted integration (53 and 92) generate a 1.0 kb band as well. (B) PCR products derived from the targeted allele were sequenced to demonstrate that the endogenous mutant STAG2 gene had been corrected via homologous recombination.

FIG. 20: Targeting strategy for correction of STAG2 mutation in H4 cells and introduction of nonsense mutation in HCT116 cells. (A) Diagram depicting the targeted homologous recombination event for correcting the endogenous STAG2 25-bp insertion causing a frameshift at codon 357 in H4 cells. In the initial step, an AAV-based targeting vector was created for the purpose of correcting the exon 12 mutation, leaving behind a FLOXed splice acceptor-IRES-Neo^{R} gene in intron 13. Clones with targeted integration and mutation correction were identified by PCR and DNA sequencing. These cells (pre-Cre clones) were then transiently infected with a Cre-expressing adenovirus, and completed STAG2 knock-in (KI) clones in which the splice acceptor-IRES-Neo^{R} gene had been deleted by Cre/LoxP recombination were identified by screening for G418-sensitivity (post-Cre clones). (B) Diagram depicting the targeted homologous recombination event for introducing a nonsense mutation into codon 6 of the STAG2 gene in HCT116 cells. In the initial step, an AAV-based targeting vector was created for the purpose of introducing the mutation into exon 3, leaving behind a FLOXed splice acceptor-IRES-Neo^{R} gene in intron 3. Clones with targeted integration and introduction of the mutation were identified by PCR and DNA sequencing.

FIG. 21: Western blot confirmation of H4 and HCT116 STAG2 gene targeted cells. (A) H4 parental cells and two pre-Cre clones in which the STAG2 KI vector had integrated by homologous recombination but in which the splice acceptor-IRES-Neo^{R} had not yet been removed fail to express STAG2 protein. The pre-Cre clones fail to express STAG2 protein due to aberrant splicing between STAG2 exon 12 and the splice acceptor-IRES-Neo^{R}. Six STAG2 KI post-Cre clones in which the splice acceptor-IRES-Neo^{R} was successfully removed via Cre/LoxP recombination express physiologic levels of corrected STAG2 protein, comparable to the levels in C33A and HeLa cells with unmodified wild-type STAG2 alleles. (B) Parental HCT116 cells and three clones in which the STAG2 KO vector integrated randomly (non recombinants) express physiologic levels of STAG2 protein, comparable to DLD-1 cells. In contrast, four clones in which the STAG2 KO vector integrated via homologous recombination, introducing a nonsense mutation into codon 6, demonstrate abrogation of STAG2 expression. In these clones, a small amount of STAG2 appears to be expressed, presumably via re-initiation at a downstream methionine such as amino acid 70.

FIG. 22: Global gene expression profiling of isogenic sets of STAG2 gene targeted cells. (A) Affymetrix GeneChip Human Gene 1.0 ST arrays were used to generate gene expression profiles in parental H4 cells, two independently derived non-recombinant clones, and three independently derived STAG2 KI post-Cre clones. Composite expression profiles of the three STAG2-mutant cells were plotted against the composite expression profiles of the three STAG2-corrected cells. (B) Identical composite expression profile comparison as in (A) using two non-recombinant HCT116 clones and two HCT116 STAG2 KO clones.

FIG. 23: Karyotypic analysis of STAG2-proficient and deficient cells. Isogenic STAG2-proficient and deficient cells were arrested in mitosis and karyotypes prepared using Wright's Stain. Chromosomes were counted in 100 metaphase cells for each cell line to determine the diversity of chromosome counts within the cell population. Chromosome counts are shown for STAG2-proficient and deficient H4 cells (A), 42MGBA cells (B), and HCT116 cells (C). Distribution curves derived from this data are depicted in FIG. 4(D-E).

### DETAILED DESCRIPTION OF THE INVENTION

### Nucleic Acids and Proteins

As used herein, the terms “nucleic acid”, “polynucleotide”, “polynucleotide molecule”, “polynucleotide sequence” and plural variants are used interchangeably to refer to a wide variety of molecules, including single strand and double strand DNA and RNA molecules, cDNA sequences, genomic DNA sequences of exons and introns, chemically synthesized DNA and RNA sequences, and sense strands and corresponding antisense strands. Polynucleotides of the invention may also comprise known analogs of natural nucleotides that have similar properties as the reference natural nucleic acid.

As used herein, the terms “polypeptide”, “protein” and plural variants are used interchangeably and refer to a compound made up of a single chain of amino acids joined by peptide bonds. Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imbenzylhistidine.

An exemplary STAG2 polynucleotide of the invention is set forth as SEQ ID NO: 1, which is the human stromal antigen 2 (STAG2) transcript variant 1 mRNA (GenBank® Accession No. NM_{—}001042479). SEQ ID NO: 2 is a portion of SEQ ID NO: 1 covering the last 21 nucleotides of the ninth intron and the first 9 nucleotides of the tenth exon. SEQ ID NO: 5 is a portion of SEQ ID NO: 1 covering the last 10 nucleotides of the eleventh intron and the first 20 nucleotides of the twelfth exon. Exemplary STAG2 polypeptides of the invention include variants of SEQ ID NO: 6 that are described in Table 1. SEQ ID NO: 6 is the STAG2 isoform a encoded by SEQ ID NO: 1.

Nucleic acids of the invention also comprise nucleic acids complementary to SEQ ID NO: 1 and subsequences and elongated sequences of SEQ ID NO: 1 and complementary sequences thereof. Complementary sequences are two nucleotide sequences that comprise antiparallel nucleotide sequences capable of pairing with one another upon formation of hydrogen bonds between base pairs. Like other polynucleotides in accordance with the present invention, complementary sequences maybe substantially similar to one another as described previously. A particular example of a complementary nucleic acid segment is an antisense oligonucleotide.

An elongated sequence is one in which nucleotides (or other analogous molecules) are added to a nucleic acid sequence. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, introns, additional restriction enzyme sites, multiple cloning sites, and other coding segments. Thus, the present invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

A subsequence is a sequence of nucleic acids that comprises a part of a longer nucleic acid sequence. An exemplary subsequence is a probe or a primer. Conditions under which a nucleic acid probe or primer will typically hybridize to its target sequence but to no other sequences when that sequence is present in a complex nucleic acid mixture (e.g., total cellular DNA or RNA) are stringent in nature. In the context of nucleic acid hybridization experiments such as Southern and Northern blot analyses, stringent hybridization conditions and stringent hybridization wash conditions are both sequence- and environment-dependent. An extensive guide to the hybridization of nucleic acids is found in Tijssen, Laboratory Techniques in Biochemistry and Molecular Biology-Hybridization with Nucleic Acid Probes, part I chapter 2, Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. Another example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An exemplary medium stringency wash for a duplex of, e.g., more than 100 nucleotides, is 1×SSC at 45° C. for 15 minutes. An example low stringency wash for a duplex of, e.g., more than 100 nucleotides, is 4×-6×SSC at 40° C. for 15 minutes. For short probes (e.g., about 10 to 50 nucleotides), stringent conditions typically involve salt concentrations of less than about 1.0 M sodium ions, typically about 0.01 to 1.0 M sodium ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention. A substantially identical nucleotide sequence preferably hybridizes to a reference nucleotide sequence in 7% sodium dodecyl sulfate (SDS), 0.5 M NaPO₄, 1 mM EDTA at 50° C. with washing in 2×SSC, 0.1% SDS at 50° C., more preferably in 7% sodium dodecyl sulfate (SDS), 0.5 M NaPO₄, 1 mM EDTA at 50° C. with washing in 1×SSC, 0.1% SDS at 50° C., still more preferably in 7% sodium dodecyl sulfate (SDS), 0.5 M NaPO₄, 1 mM EDTA at 50° C. with washing in 0.5×SSC, 0.1% SDS at 50° C., even more preferably in 7% sodium dodecyl sulfate (SDS), 0.5 M NaPO₄, 1 mM EDTA at 50° C. with washing in 0.1×SSC, 0.1% SDS at 50° C., and most preferably in 7% sodium dodecyl sulfate (SDS), 0.5 M NaPO₄, 1 mM EDTA at 50° C. with washing in 0.1×SSC, 0.1% SDS at 65° C.

Polynucleotides of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art (see e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/New York; Ausubel (ed.) Short Protocols in Molecular Biology, 3rd ed., 1995, Wiley, New York).

Isolated polypeptides of the invention may be purified and characterized using a variety of standard techniques that are known to the skilled artisan (see e.g., Schroder et al., The Peptides, 1965, Academic Press, New York; Bodanszky, Principles of Peptide Synthesis, 2nd rev. ed. 1993, Springer-Verlag, Berlin/New York; Ausubel (ed.), Short Protocols in Molecular Biology, 3rd ed., 1995, Wiley, New York).

The present invention also encompasses methods for detecting a nucleic acid molecule that encodes a STAG2 protein. Such methods may be used to detect gene variants or altered gene expression. Sequences detected by methods of the invention may detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a STAG2 nucleic acid molecule may be measured, for example, using an RT-PCR assay (see e.g., Chiang, J. Chromatogr. A., 806:209-218 (1998) and references cited therein).

The present invention also encompasses genetic assays using STAG2 nucleic acids for quantitative trait loci (QTL) analysis and to screen for genetic variants, for example by allele-specific oligonucleotide (ASO) probe analysis (Conner et al., Proc. Natl. Acad. Sci. USA, 80(1):278-282 (1983)), oligonucleotide ligation assays (OLAs) (Nickerson et al., Proc. Natl. Acad. Sci. USA, 87(22):8923-8927 (1990)), single-strand conformation polymorphism (SSCP) analysis (Orita et al., Proc. Natl. Acad. Sci. USA, 86(8):2766-2770 (1989)), SSCP/heteroduplex analysis, enzyme mismatch cleavage, direct sequence analysis of amplified exons (Kestila et al., Mol. Cell, 1(4):575-582 (1998); Yuan et al., Hum. Mutat., 14(5):440-446 (1999)), allele-specific hybridization (Stoneking et al., Am. J. Hum. Genet., 48(2):370-382 (1991)), and restriction analysis of amplified genomic DNA containing the specific mutation. Automated methods may also be applied to large-scale characterization of single nucleotide polymorphisms (Wang et al., Am. J. Physiol., 1998, 274(4 Pt 2):H1132-1140 (1992); Brookes, Gene, 234(2):177-186 (1999)). Preferred detection methods are non-electrophoretic, including, for example, the TAQMAN™ allelic discrimination assay, PCR-OLA, molecular beacons, padlock probes, and well fluorescence (see Landegren et al., Genome Res., 8:769-776 (1998) and references cited therein).

The present invention also encompasses methods for detecting a STAG2 polypeptide. Such methods can be used, for example, to determine levels of protein expression and correlate the level of expression with the presence or change in phenotype or level of expression in a different gene or gene product. In certain embodiments, the method involves an immunochemical reaction with an antibody that specifically recognizes a protein. Techniques for detecting such antibody-antigen conjugates or complexes are known in the art and include but are not limited to centrifugation, affinity chromatography and other immunochemical methods (see e.g., Ishikawa, Ultrasensitive and Rapid Enzyme Immunoassay, 1999, Elsevier, Amsterdam/New York, United States of America; Law, Immunoassay: A Practical Guide, 1996, Taylor & Francis, London/Bristol, Pennsylvania, United States of America; Liddell et al., Antibody Technology, 1995, Bios Scientific Publishers, Oxford, United Kingdom; and references cited therein).

### STAG2 Expression Systems

An expression system refers to a host cell comprising a heterologous nucleic acid and the protein encoded by the heterologous nucleic acid. For example, a heterologous expression system may comprise a host cell transfected with a construct comprising a STAG2 nucleic acid encoding a protein operatively linked to a promoter, or a cell line produced by introduction of STAG2 nucleic acids into a host cell genome. The expression system may further comprise one or more additional heterologous nucleic acids relevant to STAG2 function, such as targets of STAG2 transcriptional activation or repression activity. These additional nucleic acids may be expressed as a single construct or multiple constructs.

A construct for expressing a STAG2 protein may include a vector sequence and a STAG2 nucleotide sequence, wherein the STAG2 nucleotide sequence is operatively linked to a promoter sequence. A construct for recombinant STAG2 expression may also comprise transcription termination signals and sequences required for proper translation of the nucleotide sequence. Constructs may also contain a ‘signal sequence’ or ‘leader sequence’ to facilitate co-translational or post-translational transport of the peptide of interest to certain intracellular structures such as the endoplasmic reticulum or Golgi apparatus. Constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence. Preparation of an expression construct, including addition of translation and termination signal sequences, is known to one skilled in the art.

The promoter may be any polynucleotide sequence that shows transcriptional activity in the host cell. The promoter may be native or analogous, or foreign or heterologous, to the host cell and/or to the DNA sequence of the invention. Where the promoter is native or endogenous to the host cell, it is intended that the promoter is found in the cell into which the promoter is introduced. Where the promoter is foreign or heterologous to the DNA sequence of the invention, the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley et al., Nucleic Acids Res., 15:2343-61 (1987). Also, the location of the promoter relative to the transcription start may be optimized (see e.g., Roberts et al., Proc. Natl. Acad. Sci. USA, 76:760-4 (1979)). Many suitable promoters for use in human cell lines are well known in the art. The promoter may include, or be modified to include, one or more enhancer elements to thereby provide for higher levels of transcription. Where appropriate, the vector and STAG2 sequences may be optimized for increased expression in the transformed host cell. That is, the sequences can be synthesized using host cell-preferred codons for improving expression, or may be synthesized using codons at a host-preferred codon usage frequency.

### Host Cells

Host cells are cells into which a heterologous nucleic acid molecule of the invention may be introduced. Preferred host cells for functional assays comprise paired (i.e., otherwise isogenic) STAG2-proficient and deficient cell lines. Examples include H4 and H4 STAG2 KI post-Cre 8-1 paired cell lines and HCT116 and HCT116 STAG2 KO 7 paired cell lines.

A host cell line may be chosen which modulates the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed.

The present invention further encompasses recombinant expression of a STAG2 protein in a stable cell line. Methods for generating a stable cell line following transformation of a heterologous construct into a host cell are known in the art (see e.g., Joyner, Gene Targeting: A Practical Approach, 1993, Oxford University Press, Oxford/New York). Thus, transformed cells and tissues are understood to encompass not only the end product of a transformation process, but also transgenic progeny or propagated forms thereof.

### STAG2 Inhibitors

The present invention further discloses assays to identify STAG2 binding partners and STAG2 inhibitors. STAG2 antagonists/inhibitors are agents that alter chemical and biological activities or properties of a STAG2 protein. Methods of identifying inhibitors involve assaying a reduced level or quality of STAG2 function in the presence of one or more agents. Exemplary STAG2 inhibitors include small molecules as well as biological inhibitors as described herein below.

As used herein, the term “agent” refers to any substance that potentially interacts with a STAG2 nucleic acid or protein, including any of synthetic, recombinant, or natural origin. An agent suspected to interact with a protein may be evaluated for such an interaction using the methods disclosed herein.

Exemplary agents include but are not limited to peptides, proteins, nucleic acids, small molecules (e.g., chemical compounds), antibodies or fragments thereof, nucleic acid protein fusions, any other affinity agent, and combinations thereof. An agent to be tested may be a purified molecule, a homogenous sample, or a mixture of molecules or compounds.

A small molecule refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Exemplary nucleic acids that may be used to disrupt STAG2 function include antisense RNA and small interfering RNAs (siRNAs). These inhibitory molecules may be prepared based upon the STAG2 gene sequence and known features of inhibitory nucleic acids.

Agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of agents in a library may be assayed simultaneously. Optionally, agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667988), a library of nucleic acid-protein fusions (U.S. Pat. No. 6,214,553), and a library of any other affinity agent that may potentially bind to a STAG2 protein.

A library may comprise a random collection of molecules. Alternatively, a library may comprise a collection of molecules having a bias for a particular sequence, structure, or conformation, for example, as for inhibitory nucleic acids (see e.g., U.S. Pat. Nos. 5,264,563 and 5,824,483). Methods for preparing libraries containing diverse populations of various types of molecules are known in the art, for example as described in U.S. patents cited herein above. Numerous libraries are also commercially available.

A control level or quality of STAG2 activity refers to a level or quality of wild type STAG2 activity. When evaluating the inhibiting capacity of an agent, a control level or quality of STAG2 activity comprises a level or quality of activity in the absence of the agent. A control level may also be established by a phenotype or other measureable parameter.

Methods of identifying STAG2 inhibitors also require that the inhibiting capacity of an agent be assayed. Assaying the inhibiting capacity of an agent may comprise determining a level of STAG2 gene expression; determining DNA binding activity of a recombinantly expressed STAG2 protein; determining an active conformation of a STAG2 protein; or determining a change in response to binding of a STAG2 inhibitor. The inhibiting capacity of an agent may also be identified by administering the agent to a sample comprising at least one cell comprising a STAG2 polynucleotide or polypeptide associated with at least one chromosomal aberration and determining whether the agent affects the viability of the at least one cell. In particular embodiments, a method of identifying a STAG2 inhibitor may comprise (a) providing a paired set of host cells expressing two different STAG2 proteins; (b) contacting the host cells with an agent; (c) examining the host cells for a difference in response to the agent (e.g., survivability); and (d) selecting an agent that induces the difference in response. For example, the first sample may comprise one or more homozygous STAG2 deficient cells (e.g., H4 STAG2 KI post-Cre 8-1 cells or HCT116 STAG2 KO 7 cell) and the second sample may comprise a like quantity of isogenic STAG2-proficient cells (e.g., H4 cells or HCT116 cells respectively).

Any of the agents so identified in the inhibitory or binding assays disclosed hereinafter may be subsequently applied to a different cell as desired to effectuate the same or similar change in that cell. In cases where the agent selectively impacts the viability of a cell comprising a STAG2 polynucleotide or polypeptide associated with at least one chromosomal aberration, the agent may be suitable for use in the treatment of cancers associated with expression of that STAG2 polynucleotide or polypeptide.

The present invention also encompasses a rapid and high throughput screening method that relies on the methods described herein. This screening method comprises separately contacting a STAG2 protein with a plurality of agents.

The in vitro and cellular assays of the invention may comprise soluble assays, or may further comprise a solid phase substrate for immobilizing one or more components of the assay. For example, a STAG2 protein, or a cell expressing a STAG2 protein, may be bound directly to a solid state component via a covalent or non-covalent linkage. Optionally, the binding may include a linker molecule or tag that mediates indirect binding of a STAG2 protein to a substrate.

### STAG2 Binding Assays

The present invention also encompasses methods of identifying of a STAG2 inhibitor by determining specific binding of a substance (e.g., an agent described previously) to a STAG2 protein. For example, a method of identifying a STAG2 binding partner may comprise: (a) providing a STAG2 protein; (b) contacting the STAG2 protein with one or more agents under conditions sufficient for binding; (c) assaying binding of the agent to the isolated STAG2 protein; and (d) selecting an agent that demonstrates specific binding to the STAG2 protein. Specific binding may also encompass a quality or state of mutual action such that binding of an agent to a STAG2 protein is inhibitory.

Specific binding refers to a binding reaction which is determinative of the presence of the protein in a heterogeneous population of proteins and other biological materials. The binding of an agent to a STAG2 protein may be considered specific if the binding affinity is about 1×10⁴ M⁻¹ to about 1×10⁶ M⁻¹ or greater. Specific binding also refers to saturable binding. To demonstrate saturable binding of an agent to a STAG2 protein, Scatchard analysis may be carried out as described, for example, by Mak et al., J. Biol. Chem., 264:21613-21618 (1989).

Several techniques may be used to detect interactions between a STAG2 protein and an agent without employing a known competitive inhibitor. Representative methods include, but are not limited to, Fluorescence Correlation Spectroscopy, Surface-Enhanced Laser Desorption/Ionization Time-Of-Flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a STAG2 protein) is expressed as a recombinant protein in a host cell with a sequence tag, such as a poly-histidine sequence, inserted at the N-terminus or C-terminus. The protein is purified using chromatographic methods. For example, the poly-histidine tag may be used to bind the expressed protein to a metal chelate column such as Ni²⁺ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756 (1998)). In a typical experiment, a target protein (e.g., a STAG2 protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a STAG2 protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction (see also Homola et al., Sensors and Actuators, 54:3-15 (1999) and references cited therein).

### Conformational Assays

The present invention also encompasses methods of identifying STAG2 binding partners and inhibitors that rely on a conformational change of a STAG2 protein when bound by or otherwise interacting with a substance (e.g., an agent described previously). For example, application of circular dichroism to solutions of macromolecules reveals the conformational states of these macromolecules. The technique may distinguish random coil, alpha helix, and beta chain conformational states.

To identify inhibitors of a STAG2 protein, circular dichroism analysis may be performed using a recombinantly expressed STAG2 protein. A STAG2 protein is purified, for example by ion exchange and size exclusion chromatography, and mixed with an agent. The mixture is subjected to circular dichroism. The conformation of a STAG2 protein in the presence of an agent is compared to a conformation of a STAG2 protein in the absence of the agent. A change in conformational state of a STAG2 protein in the presence of an agent identifies a STAG2 binding partner or inhibitor. Representative methods are described in U.S. Pat. Nos. 5,776,859 and 5,780,242. Antagonistic activity of the inhibitor may be assessed using functional assays, such assaying nitrate content, nitrate uptake, lateral root growth, or plant biomass, as described herein.

In accordance with the disclosed methods, cells expressing STAG2 may be provided in the form of a kit useful for performing an assay of STAG2 function. For example, a kit for detecting a STAG2 may include cells transfected with DNA encoding a full-length STAG2 protein and a medium for growing the cells.

Assays of STAG2 activity that employ transiently transfected cells may include a marker that distinguishes transfected cells from non-transfected cells. A marker may be encoded by or otherwise associated with a construct for STAG2 expression, such that cells are simultaneously transfected with a nucleic acid molecule encoding STAG2 and the marker. Representative detectable molecules that are useful as markers include but are not limited to a heterologous nucleic acid, a protein encoded by a transfected construct (e.g., an enzyme or a fluorescent protein), a binding protein, and an antigen.

Assays employing cells expressing recombinant STAG2 may additionally employ control cells that are substantially devoid of native STAG2 and, optionally, proteins substantially similar to a STAG2 protein. When using transiently transfected cells, a control cell may comprise, for example, an untransfected host cell. When using a stable cell line expressing a STAG2 protein, a control cell may comprise, for example, a parent cell line used to derive the STAG2-expressing cell line.

### Anti-STAG2 Antibodies

In another aspect of the invention, a method is provided for producing an antibody that specifically binds a STAG2 protein. According to the method, a full-length recombinant STAG2 protein is formulated so that it may be used as an effective immunogen, and used to immunize an animal so as to generate an immune response in the animal. The immune response is characterized by the production of antibodies that may be collected from the blood serum of the animal. An exemplary anti-STAG2 antibody is the JS-12 mouse monoclonal antibody (Santa Cruz Biotechnology, sc-81852).

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Antibodies of the invention include diabodies, tetrameric antibodies, single chain antibodies, tretravalent antibodies, multispecific antibodies (e.g., bispecific antibodies), and domain-specific antibodies that recognize a particular epitope. Cell lines that produce anti-STAG2 antibodies are also encompassed by the invention.

Specific binding of an antibody to a STAG2 protein refers to preferential binding to a STAG2 protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

STAG2 antibodies prepared as disclosed herein may be used in methods known in the art relating to the expression and activity of STAG2 proteins, e.g., for cloning of nucleic acids encoding a STAG2 protein, immunopurification of a STAG2 protein, and detecting a STAG2 protein in a sample, and measuring levels of a STAG2 protein in samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Analysis of Glioblastomas and Related Cell Lines

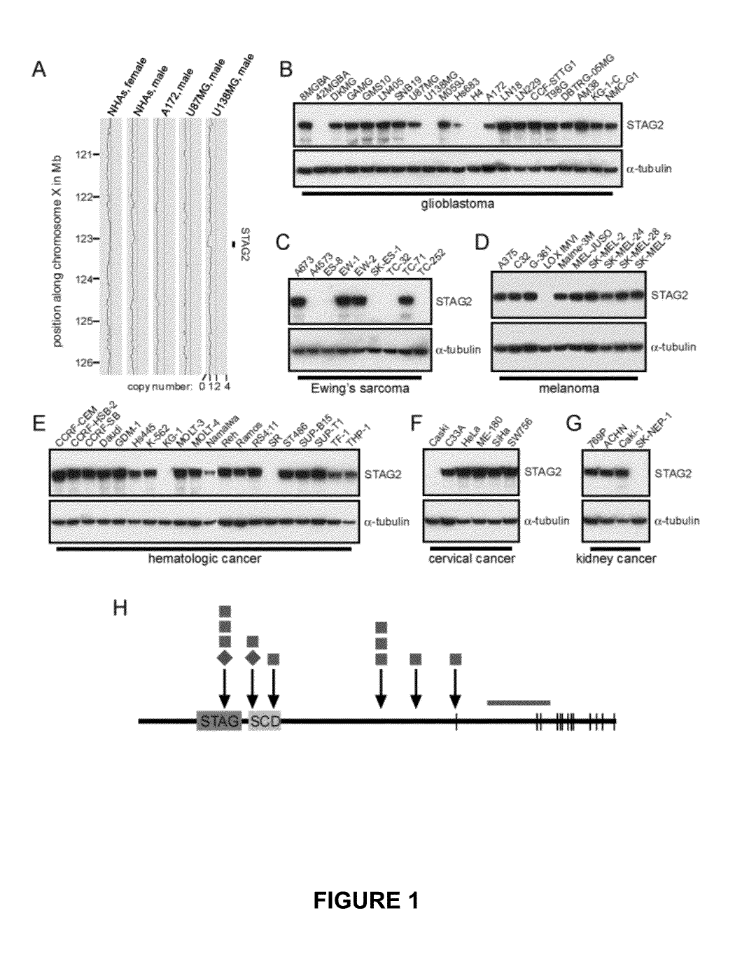

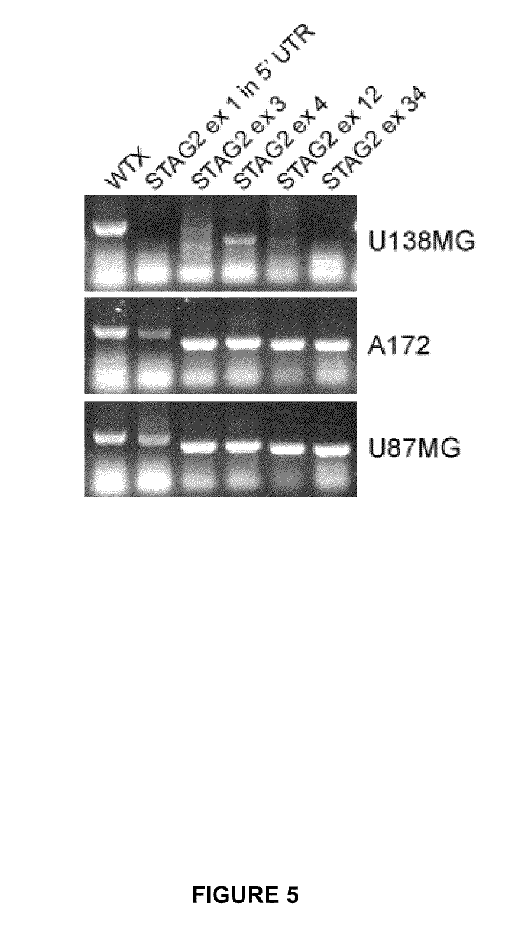

A panel of 21 glioblastoma cell lines was obtained from the American Type Culture Collection (U87MG, U138MG, M059J, Hs683, H4, A172, LN18, LN229, CCF-STTG1, T98G, DBTRG-05MG), DSMZ (8MGBA, 42MGBA, DKMG, GAMG, GMS10, LN₄O₅, SNB19), and the Japan Health Sciences Foundation Health Science Research Resources Bank (AM38, NMC-G1, KG-1-C). A copy number analysis of the panel was performed using Affymetrix 250K SNP arrays to identify novel regions of amplification and deletion. In one of these cell lines, U138MG, a region of genomic deletion on the X chromosome between 122.930-123.226 Mb was identified. This region contains the STAG2 gene (FIG. 1A). The deletion of STAG2 in U138MG cells was confirmed via PCR (FIG. 5).

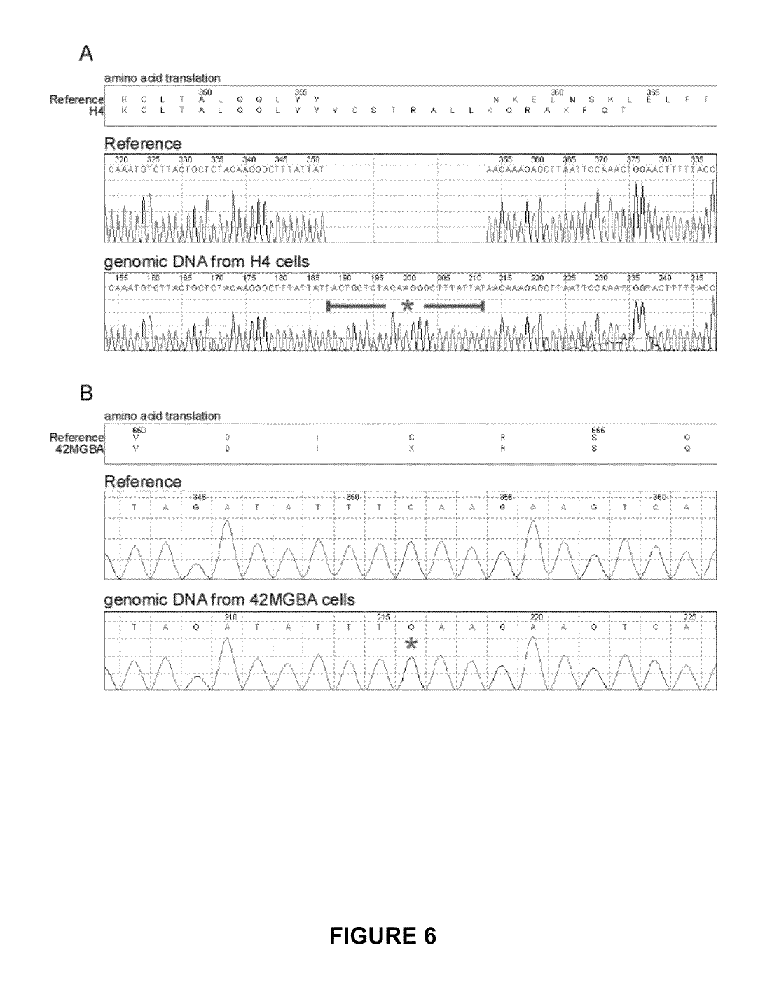

The expression of STAG2 in this panel was then evaluated by Western blot using STAG2 clone J-12 mouse monoclonal antibody (Santa Cruz Biotechnology, sc-81852) and a-tubulin Ab-2 clone DM 1A mouse monoclonal antibody (Neomarkers) (FIG. 1B). As expected, U138MG cells expressed no detectable STAG2 protein. 42MGBA and H4 cells similarly lacked STAG2 protein expression, despite no evidence of copy number loss by SNP microarray. To test whether point mutations might be responsible for the absence of STAG2 expression in 42MGBA and H4 cells, the 33 coding exons of STAG2 were sequenced in 42MGBA and H4 cell lines. A 25-bp duplication/insertion resulted in a frameshift in H4 cells and a nonsense mutation in 42MGBA cells (see Table 1 and FIG. 6). References to exons, mRNA position and protein change are based on reference to SEQ ID NO: 1.

TABLE 1

| Sample | Tumor Type | Mutation observed | |
|---|---|---|---|
| KG-1 | AML | none identified | |
| CaSki | cervical carcinoma | deletion TTTACTCTTTAAAAATAGCT (SEQ ID NO: 3) | |
| A4573 | Ewing's sarcoma | deletion A | |
| ES-8 | Ewing's sarcoma | deletion of noncoding exons in 5′ UTR | |
| SK-ES-1 | Ewing's sarcoma | C > T (nonsense) | |
| SK-NEP-1 | Ewing's sarcoma | none identified | |
| TC-252 | Ewing's sarcoma | insertion T | |
| TC-32 | Ewing's sarcoma | insertion T | |
| ES 37 | Ewing's sarcoma | A > G, 8 by upstream of initiating ATG | |
| 42MGBA | glioblastoma | C > G (nonsense) | |
| H4 | glioblastoma | insertion TACTGCTCTACAAGGGCTTTATTAT (SEQ ID NO: 4) | |
| U138MG | glioblastoma | whole gene deletion | |
| GBM p785 | glioblastoma | A > C (missense) | |
| GBM 14 | glioblastoma | G > C, splice acceptor | |
| GBM 44 | glioblastoma | deletion AA | |
| SF7300 | glioblastoma | C > T, splice acceptor | |
| SR | immunoblastic lymphoma | intragenic deletion of exons 28-30 | |
| LOX IMVI | melanoma | deletion of exons 3-35 | |
| MM 29T | melanoma | insertion TATGAA | |
| Sample | Exon no. | mRNA position | Protein change |
| KG-1 | — | — | no protein |
| CaSki | 9 | 1072(−18) . . . 1073 | 223A > truncation |
| A4573 | 25 | 2929 | 842N > frameshift |
| ES-8 | 1, 2 | 1 . . . 307 | no protein |
| SK-ES-1 | 23 | 2607 | 735Q > Stop |
| SK-NEP-1 | — | — | no protein |
| TC-252 | 20 | 2310 . . . 2311 | 636Y > frameshift |
| TC-32 | 20 | 2310 . . . 2311 | 636Y > frameshift |
| ES 37 | 3 | 397 | no protein |
| 42MGBA | 20 | 2362 | 6535 > Stop |
| H4 | 12 | 1472 . . . 1473 | 357N > frameshift |
| U138MG | 1-35 | 1 . . . 6277 | no protein |
| GBM p785 | 11 | 1300 | 299D > A |
| GBM 14 | 9 | 1072(−1) | 223A > truncation |
| GBM 44 | 9 | 1110 . . . 1111 | 236N > frameshift |
| SF7300 | 11 | 1298(−9) | undetermined |
| SR | 28-30 | 3180 . . . 3681 | deletion 925-1092 |
| LOX IMVI | 3-35 | 308 . . . 6277 | no protein |
| MM 29T | 9 | 1078 . . . 1079 | 225K > insertNM |
| Sample | Type | Mutation observed | |
| 94-10 | Bladder cancer cell line | A > G (splice acceptor) | |
| UM-UC-3 | Bladder cancer cell line | A > T, deletion A(complex indel) | |
| UM-UC-14 | Bladder cancer cell line | G > T (splice acceptor) | |
| VM-CUB-1 | Bladder cancer cell line | insertion TTATAGTATTATAATGACT (SEQ ID NO: 7) | |
| VM-CUB-3 | Bladder cancer cell line | A > G (splice acceptor) | |
| MDACC 10056 | Bladder cancer primary tumor | C > G (nonsense) | |
| MDACC 10057 | Bladder cancer primary tumor | C > T (nonsense) | |
| MDACC 10059 | Bladder cancer primary tumor | C > T (nonsense) | |
| MDACC 10076 | Bladder cancer primary tumor | C > A (nonsense) | |
| MDACC 20031 | Bladder cancer primary tumor | G > A (splice donor) | |
| MDACC 20042 | Bladder cancer primary tumor | G > C (missense) | |
| MDACC 20062 | Bladder cancer primary tumor | C > A (missense) | |
| MDACC 20093 | Bladder cancer primary tumor | C > G, deletion AGTAAAA (complex indel) | |
| MDACC 20099 | Bladder cancer primary tumor | insertion G | |
| MDACC 20107 | Bladder cancer primary tumor | deletion C | |
| MDACC 20136 | Bladder cancer primary tumor | T > C (missense) | |
| MDACC 20146 | Bladder cancer primary tumor | C > T (missense) | |
| MDACC 20167 | Bladder cancer primary tumor | G > T, T > C, deletion AT (complex indel) | |
| MDACC 20167 | Bladder cancer primary tumor | A > T (splice acceptor) | |
| MDACC 20241 | Bladder cancer primary tumor | insertion A | |
| MDACC 20241 | Bladder cancer primary tumor | insertion T | |
| MDACC 20363 | Bladder cancer primary tumor | A > G (splice acceptor) | |
| MDACC 20586 | Bladder cancer primary tumor | deletion GT | |
| MDACC 20654 | Bladder cancer primary tumor | deletion G | |
| MDACC 20711 | Bladder cancer primary tumor | G > A (splice acceptor) | |
| MDACC 20837 | Bladder cancer primary tumor | G > C (missense) | |
| MDACC 20898 | Bladder cancer primary tumor | T > G (splice donor) | |
| MDACC 21460 | Bladder cancer primary tumor | insertion A | |
| MDACC 27627 | Bladder cancer primary tumor | insertion TC | |
| JH BLD05 | Bladder cancer primary tumor | C > G (nonsense) | |
| JH BLD08 | Bladder cancer primary tumor | insertion T (splice acceptor) | |
| Sample | Exon no. * | mRNA position * | Protein change * |
| 94-10 | 22 | 2607(−2) | 699N > frameshift |
| UM-UC-3 | 29 | 3457, 3458 | 983K > truncation |
| UM-UC-14 | 21 | 2536(−1) | 676G > frameshift |
| VM-CUB-1 | 27 | 3196 . . . 3197 | 896Y > frameshift |
| VM-CUB-3 | 6 | 799(−2) | 97S > frameshift |
| MDACC 10056 | 30 | 3734 | 1075S > Stop |
| MDACC 10057 | 8 | 1156 | 216R > Stop |
| MDACC 10059 | 19 | 2287 | 593Q > Stop |
| MDACC 10076 | 6 | 800 | 97S > Stop |
| MDACC 20031 | 11 | 1527(+1) | 339K > frameshift |
| MDACC 20042 | 29 | 3557 | 1016R > T |
| MDACC 20062 | 19 | 2287 | 593Q > K |
| MDACC 20093 | 26 | 3065, 3067 . . . 3073 | 852A > G, 853 S > frameshift |
| MDACC 20099 | 16 | 1954 . . . 1955 | 482D > frameshift |
| MDACC 20107 | 30 | 3642 | 1045R > frameshift |
| MDACC 20136 | 30 | 3742 | 1078S > P |
| MDACC 20146 | 8 | 1115 | 202S > L |
| MDACC 20167 | 24 | 2809, 2810, 2811 . . . 2812 | 767V > frameshift |
| MDACC 20167 | 30 | 3564(−2) | 1018V > frameshift |
| MDACC 20241 | 8 | 1162 . . . 1163 | 218T > frameshift |
| MDACC 20241 | 15 | 1820 . . . 1821 | 437F > frameshift |
| MDACC 20363 | 7 | 896(−8) | 129G > frameshift |
| MDACC 20586 | 11 | 1464 . . . 1465 | 318M > frameshift |
| MDACC 20654 | 18 | 2166 | 552E > frameshift |
| MDACC 20711 | 22 | 2607(−1) | 699N > frameshift |
| MDACC 20837 | 11 | 1522 | 338D > H |
| MDACC 20898 | 22 | 2694(+2) | 728Q > frameshift |
| MDACC 21460 | 26 | 3163 . . . 3164 | 885I > frameshift |
| MDACC 27627 | 31 | 3934 . . . 3935 | 1142Y > frameshift |
| JH BLD05 | 34 | 4223 | 1238S > Stop |
| JH BLD08 | 5 | 634(−7 . . . −6) | 42T > frameshift |

* The exon number, mRNA, and amino acid coordinates are annotated according to the Ensembl transcript STAG2-006 (ENST00000218089).

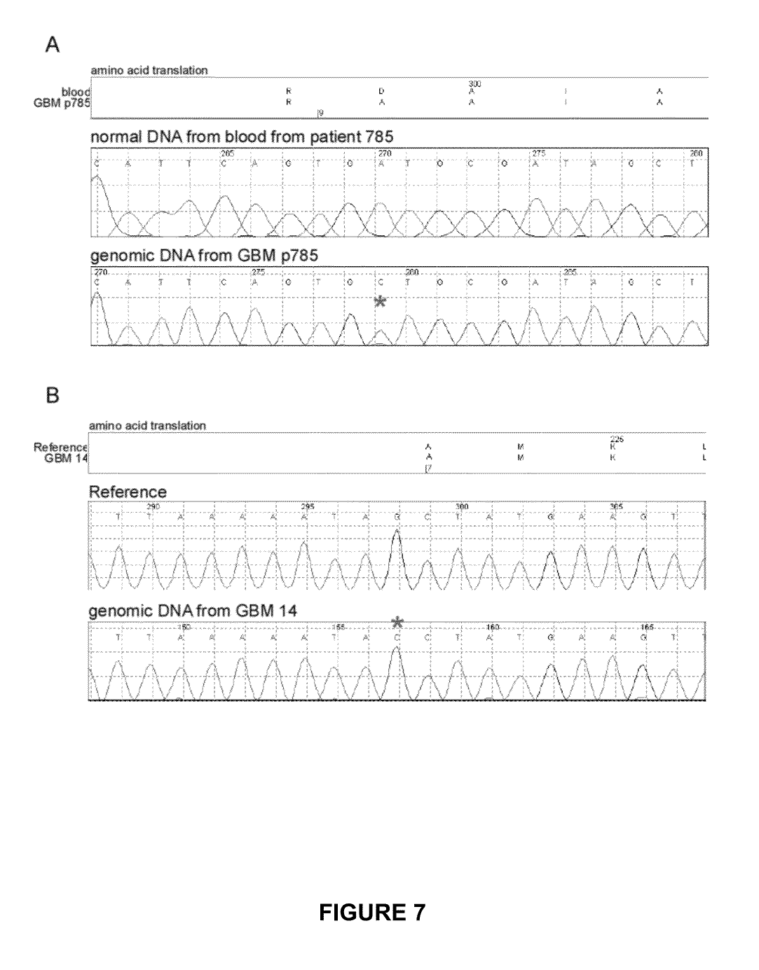

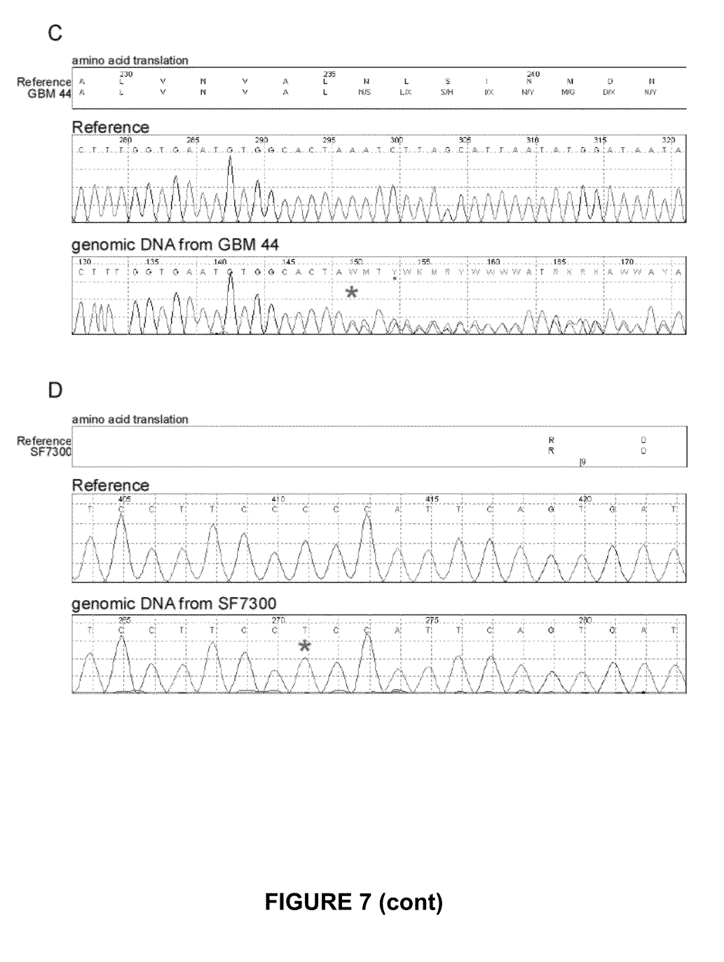

Given the relatively high frequency of genetic inactivation in glioblastoma cell lines (3/21), STAG2 was sequenced in 68 glioblastoma primary tumors and xenografts. These studies identified four additional mutations—a somatic (i.e. tumor specific) homozygous missense mutation in the stromalin conservative domain (SCD) in GBM p785, a homozygous mutation of the canonical exon 9 splice acceptor in GBM 14, a 2-bp deletion causing a frameshift in GBM 44, and a homozygous point mutation in the exon 11 splice acceptor region in GBM SF7300 (see Table 1 and FIG. 7).

### Example 2

### Analysis of Additional Tumors and Cell Lines

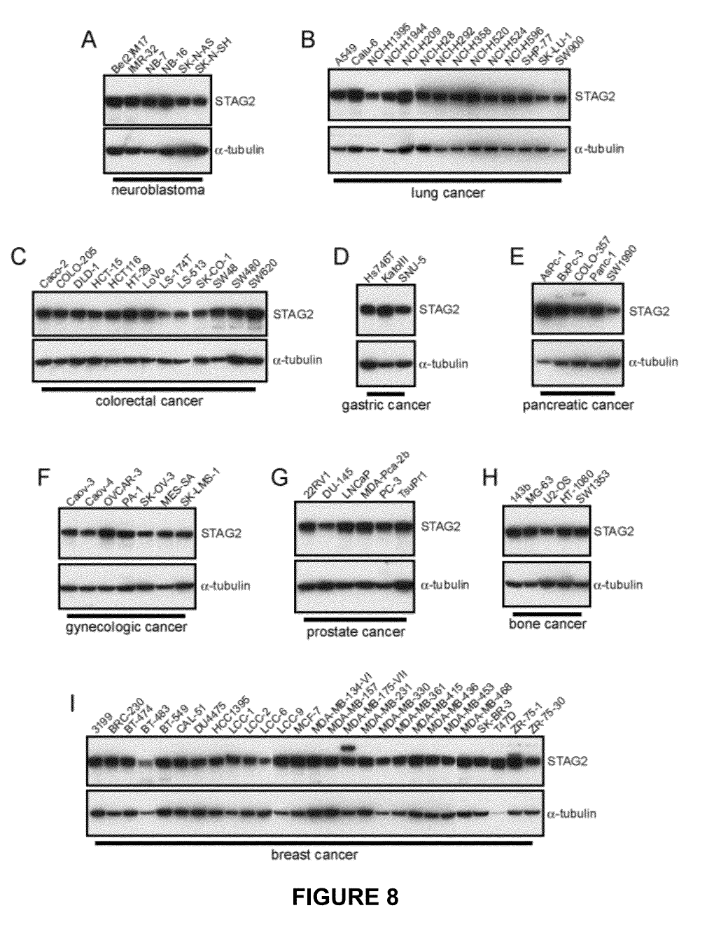

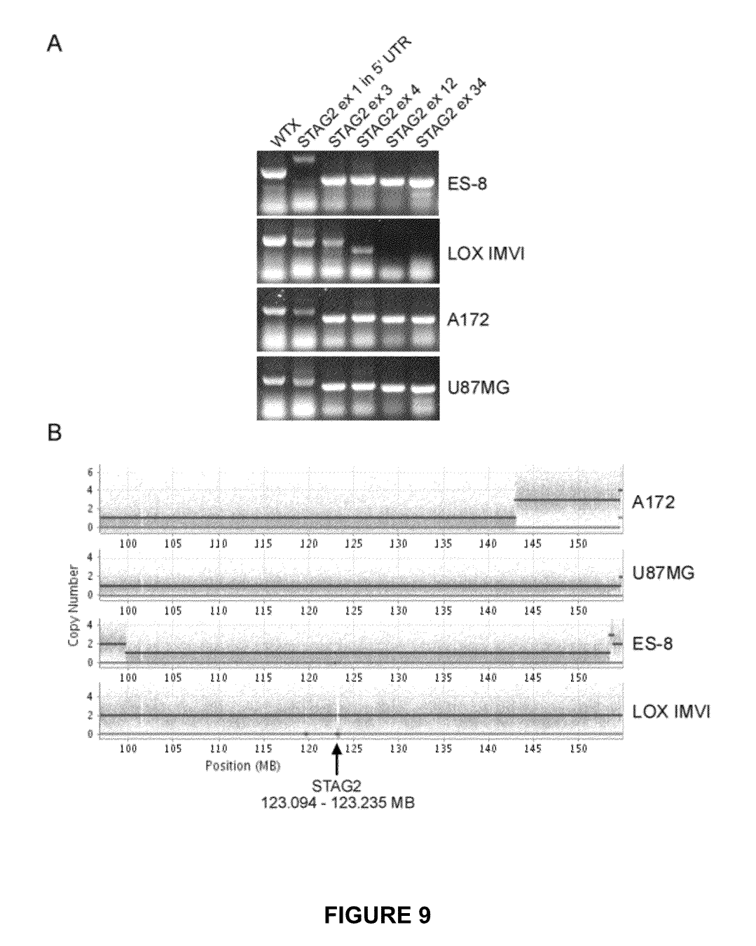

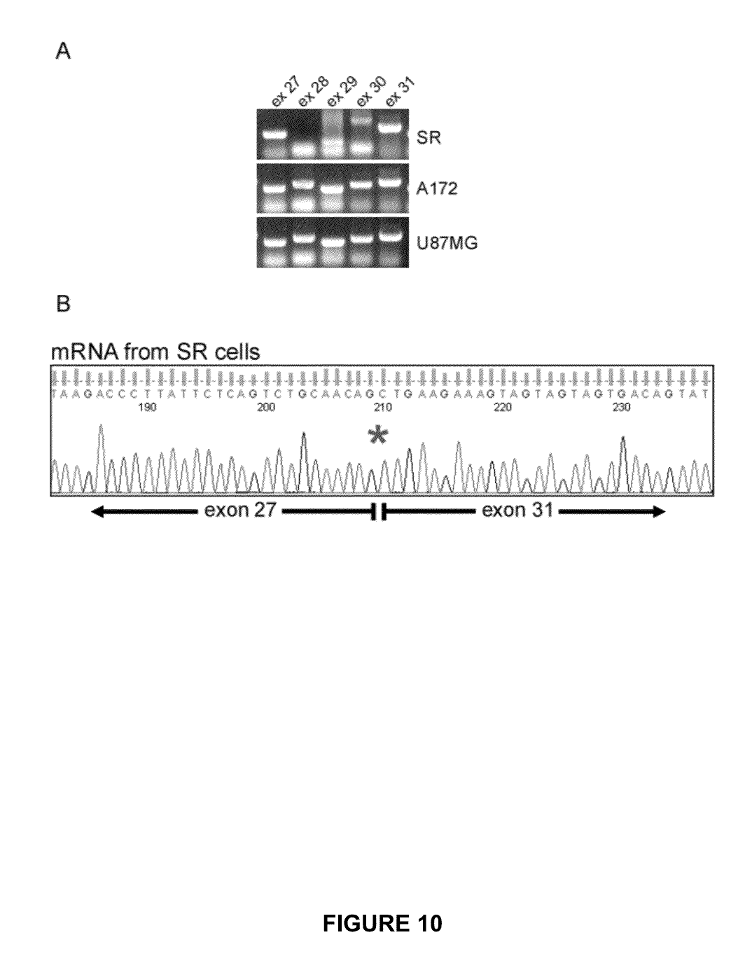

Western blots were performed on a different panel of 135 additional human cancer cell lines from a variety of tumor types. This analysis identified 10 additional cell lines that had complete absence of detectable STAG2 expression: 1/10 melanomas, 5/9 Ewing's sarcomas, 1/4 kidney cancers, 1/6 cervical cancers, and 2/20 hematologic cancers (FIG. 1C-H and FIG. 8). Sequencing of the STAG2 gene revealed genetic lesions in 8/10 of these samples—one melanoma with whole gene deletion, one immunoblastic lymphoma with intragenic deletion of multiple coding exons, one Ewing's sarcoma with deletion of the exons composing the 5′ untranslated region, one Ewing's sarcoma with a nonsense mutation, and one cervical carcinoma and three Ewing's sarcoma all with small insertion/deletion mutations causing frameshifts (see Table 1 and FIGS. 9-11).

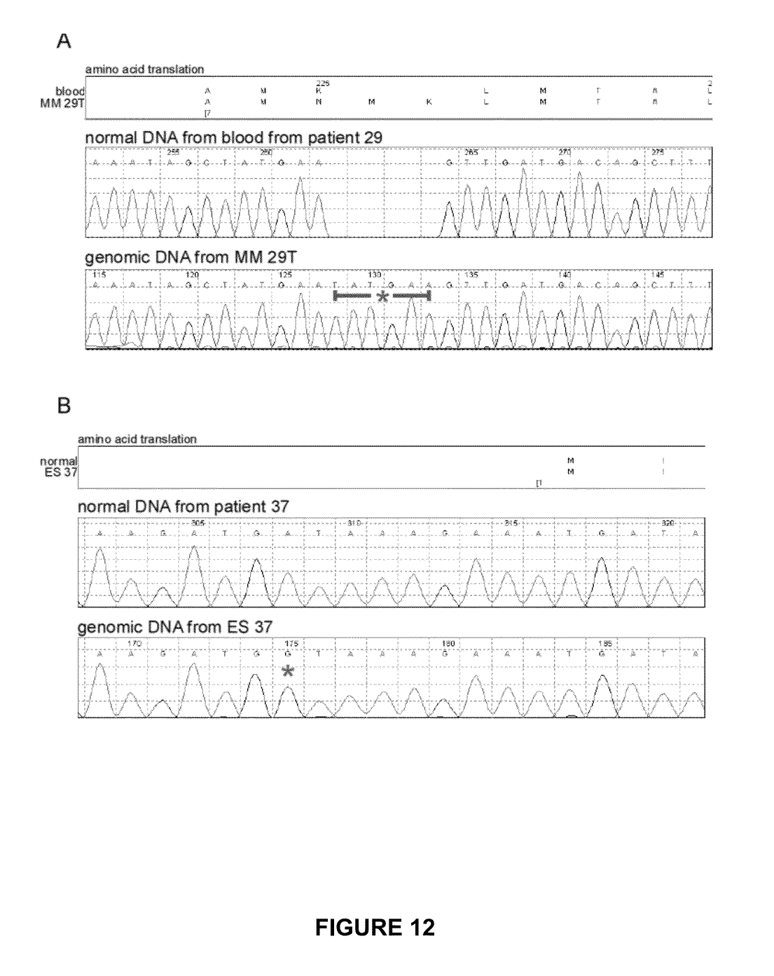

STAG2 was also sequenced in 48 melanoma and 24 Ewing's sarcoma tumors. These studies identified a somatic homozygous 6 bp insertion in the stromal antigen (STAG) domain in a melanoma sample, and a somatic homozygous point mutation 8 bp upstream of the initiating methionine in a Ewing's sarcoma sample (See Table 1 and FIG. 12).

### Example 3

### mRNA Expression

One glioblastoma and three Ewing's sarcoma samples were identified that harbored heterozygous mutations despite complete absence of STAG2 expression (see Table 2).

TABLE 2

| Sample | gDNA zygosity | mRNA zygosity | Somatic/germline | Patient age, sex | Tumor genotype | Mechanism of biallelic inactivation |
|---|---|---|---|---|---|---|
| KG-1 | — | — | ND | 59 M | XY | ND |
| CaSki | Homozygous | — | ND | 40 F | XX | loss of heterozygosity |
| A4573 | Heterozygous | Homozygous | ND | 17 F | XX | X chromosome inactivation |
| ES-8 | Homozygous | — | ND | 10 M | XY | male patient w/single allele |
| SK-ES-1 | Homozygous | — | ND | 18 M | X | male patient w/single allele |
| SK-NEP-1 | — | — | ND | 25 F | XX | ND |
| TC-252 | Heterozygous | Homozygous | ND | F | XX | X chromosome inactivation |
| TC-32 | Heterozygous | Homozygous | ND | 17 F | XX | X chromosome inactivation |
| ES 37 | Homozygous | — | somatic | 25 M | unknown | male patient |
| 42MGBA | Homozygous | — | ND | 63 M | XX | male patient w/duplicated mutant allele |
| H4 | Homozygous | — | ND | 37 M | XXYY | male patient w/duplicated mutant allele |
| U138MG | Homozygous | — | ND | 47 M | XY | male patient w/single allele |
| GBM p785 | Homozygous | — | somatic | 77 M | unknown | male patient |
| GBM 14 | Homozygous | — | ND | M | XY | male patient w/single allele |
| GBM 44 | Heterozygous | Homozygous | ND | F | XX | X chromosome inactivation |
| SF7300 | Homozygous | — | ND | M | XY | male patient w/single allele |
| SR | Homozygous | — | ND | 11 M | XY | male patient w/single allele |
| LOX IMVI | Homozygous | — | ND | 58 M | X | male patient w/single allele |
| MM 29T | Homozygous | — | somatic | 51 M | unknown | male patient |

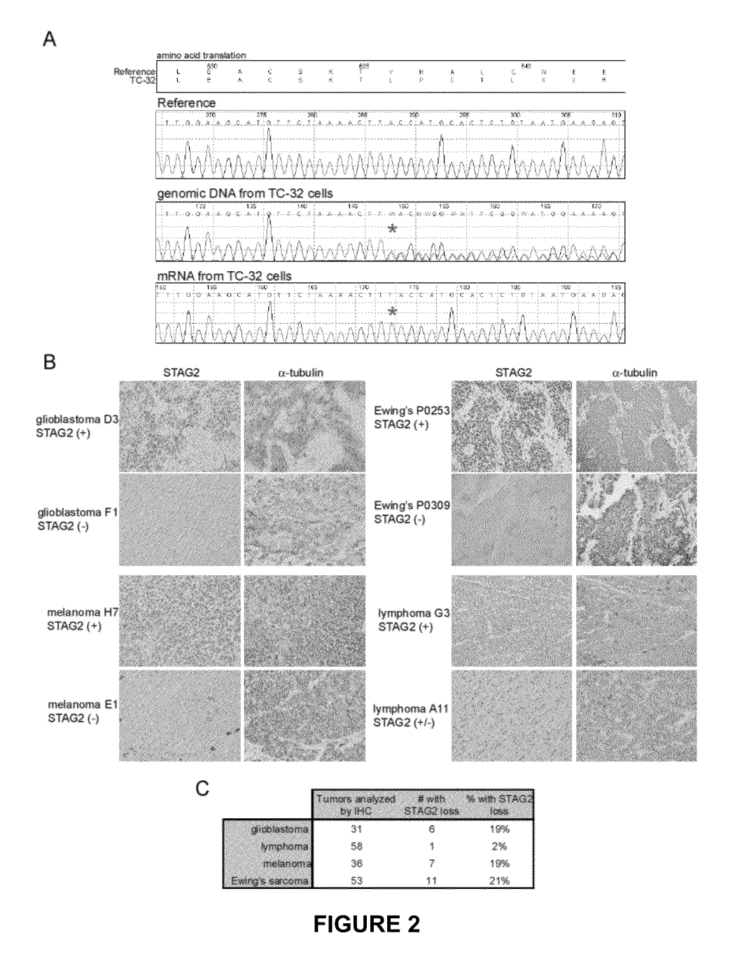

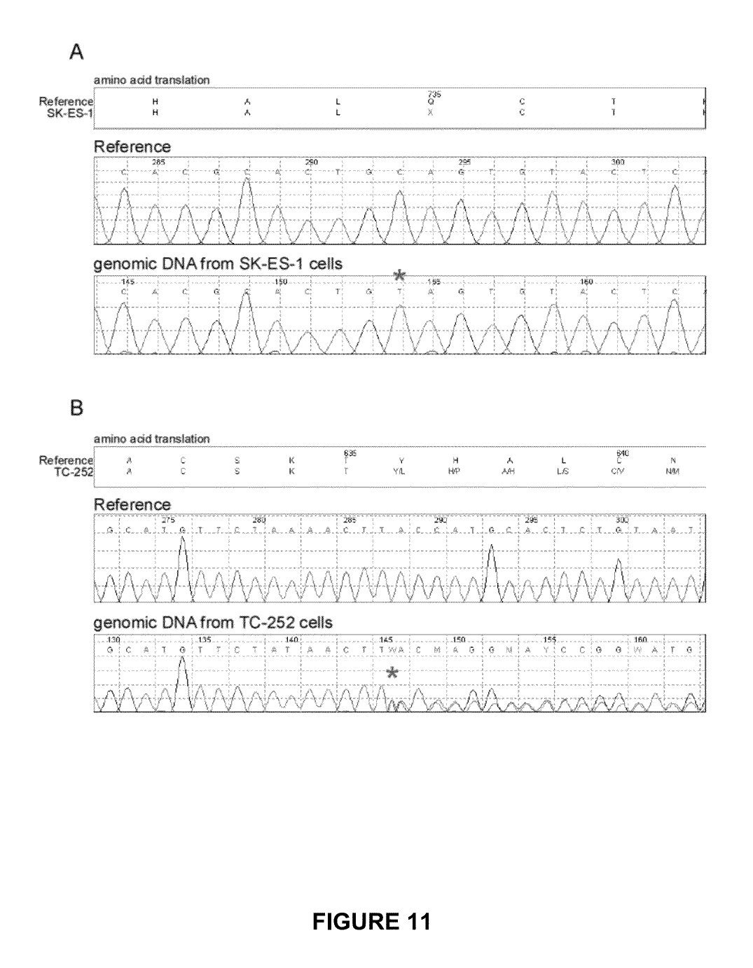

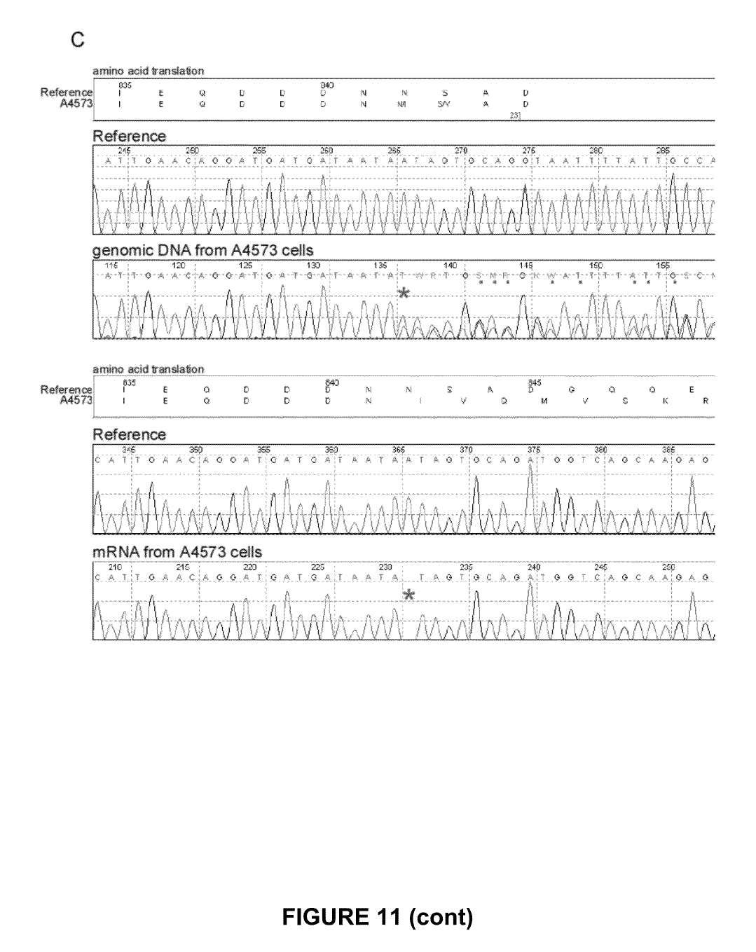

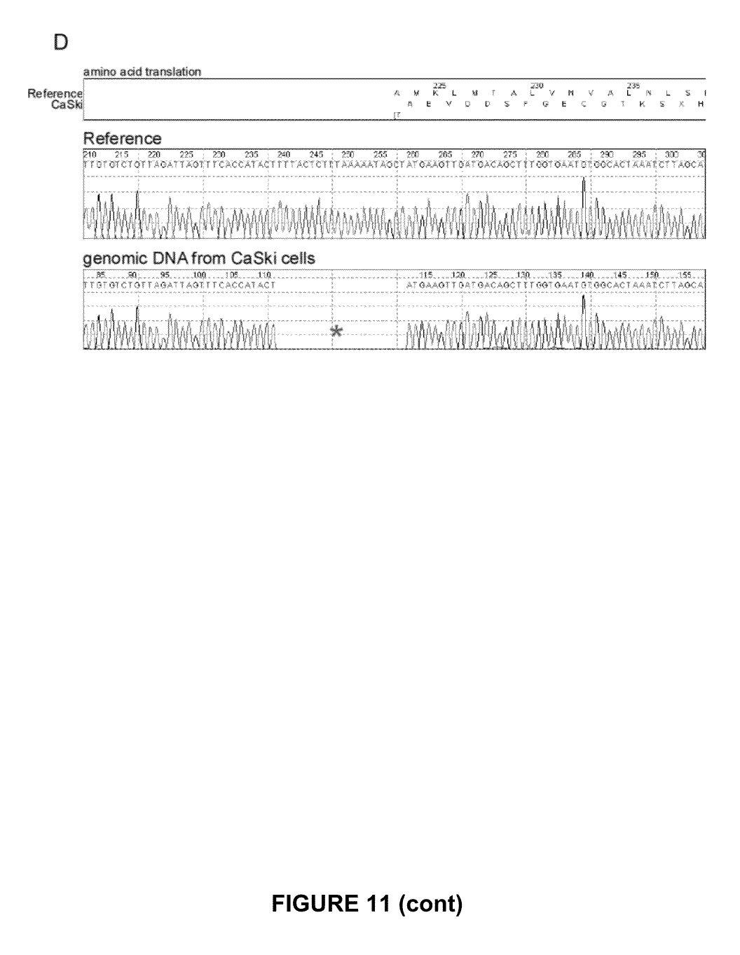

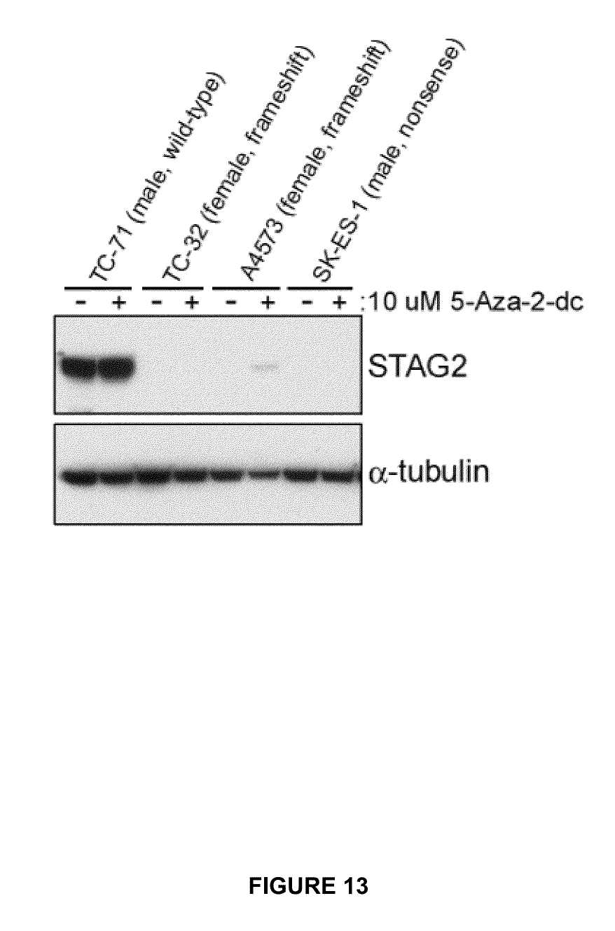

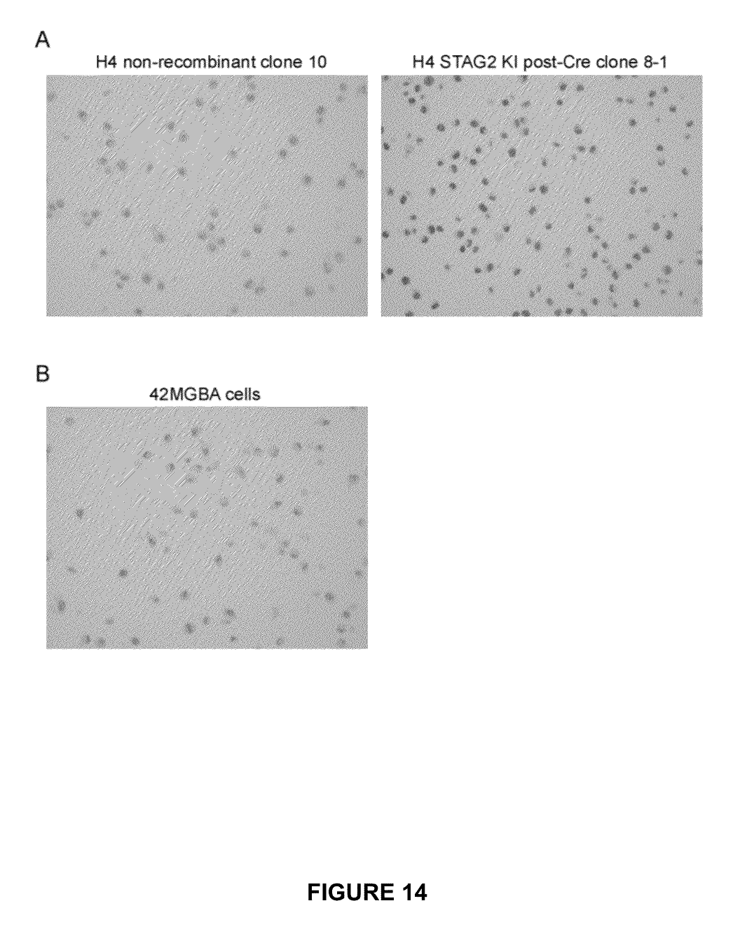

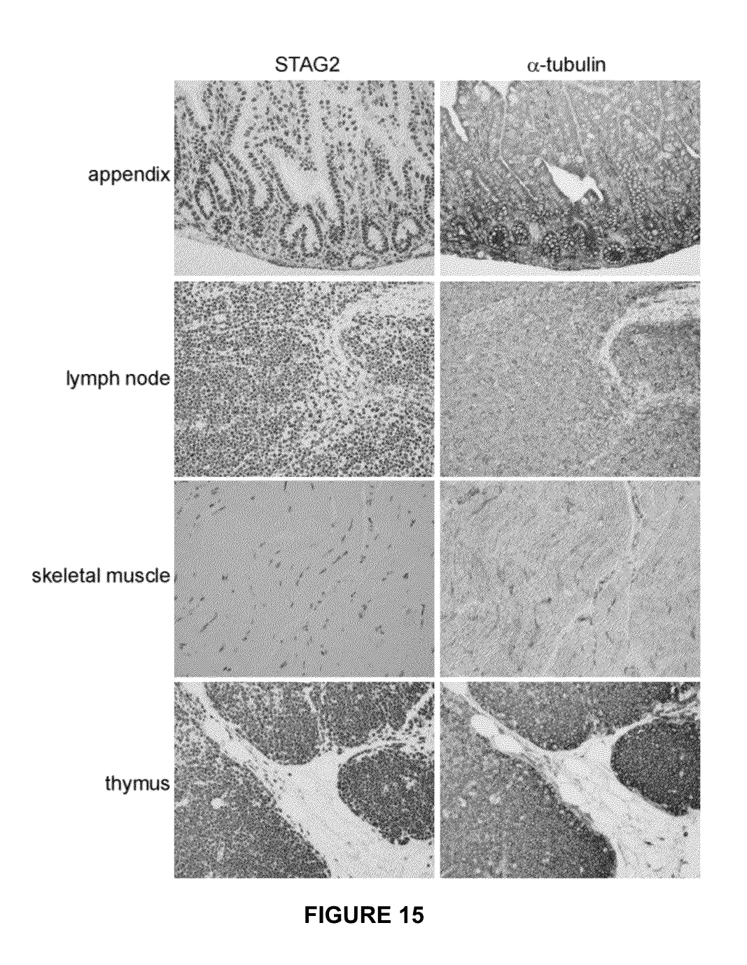

Each of these four samples was derived from a female patient, suggesting that the remaining wild-type allele of STAG2 was on the inactivated X chromosome. To provide experimental evidence for this, we sequenced STAG2 in mRNA from these four samples. Despite heterozygosity in the genomic DNA, mRNA expression was derived exclusively from the mutant STAG2 allele, thereby confirming that epigenetic inactivation of the remaining wild-type allele was the mechanism of biallelic inactivation in these samples (see Table 2 and FIGS. 2A and 11C). Expression of STAG2 in two of Ewing's sarcoma cell lines was evaluated after treatment with the DNA methylase inhibitor 5-aza-2-deoxycytidine. Treatment with this agent led to no re-expression of STAG2 from the wild-type allele in STAG2-32 cells and only minimal re-expression in A4573 cells, thereby demonstrating that X chromosome inactivation (and not promoter methylation) was indeed responsible for the “single hit” inactivation of STAG2 occurring in these tumors (see FIG. 13). This mechanism of single hit inactivation of X-linked genes in cancer has been previously reported only for the WTX gene, implicated in the pathogenesis of Wilm's tumors.

### Example 4

### Immunohistochemistry

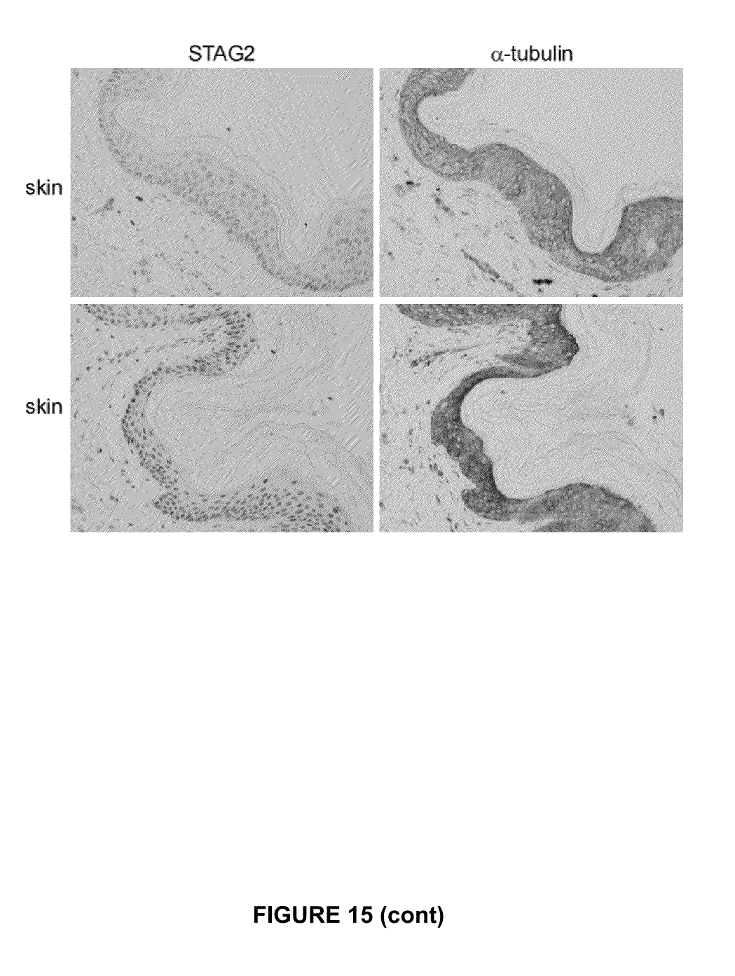

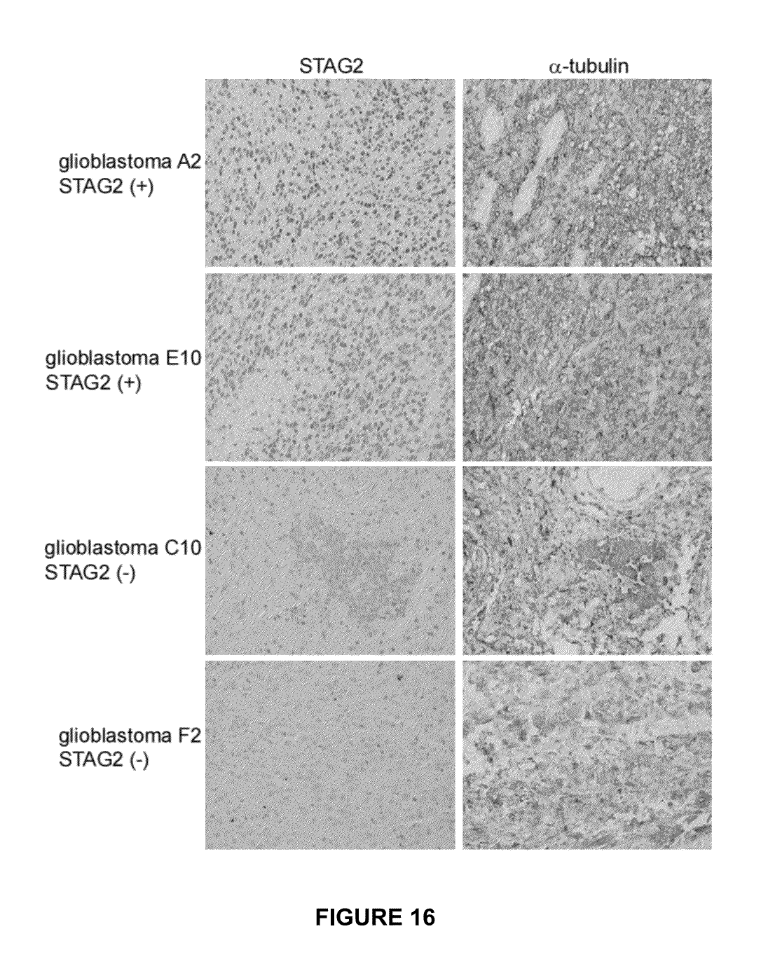

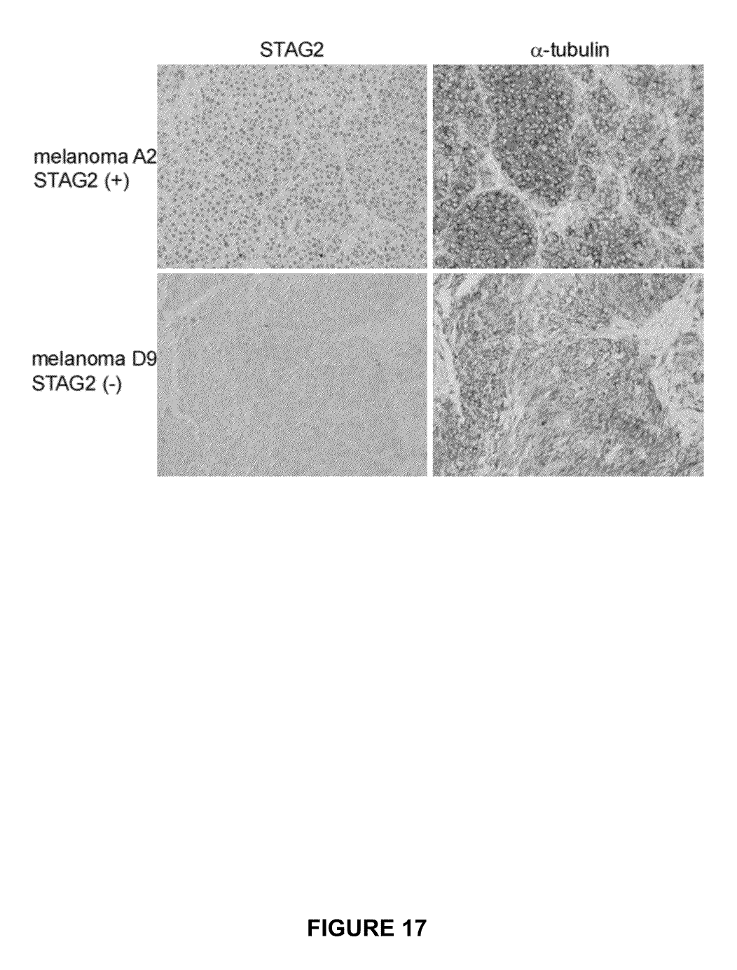

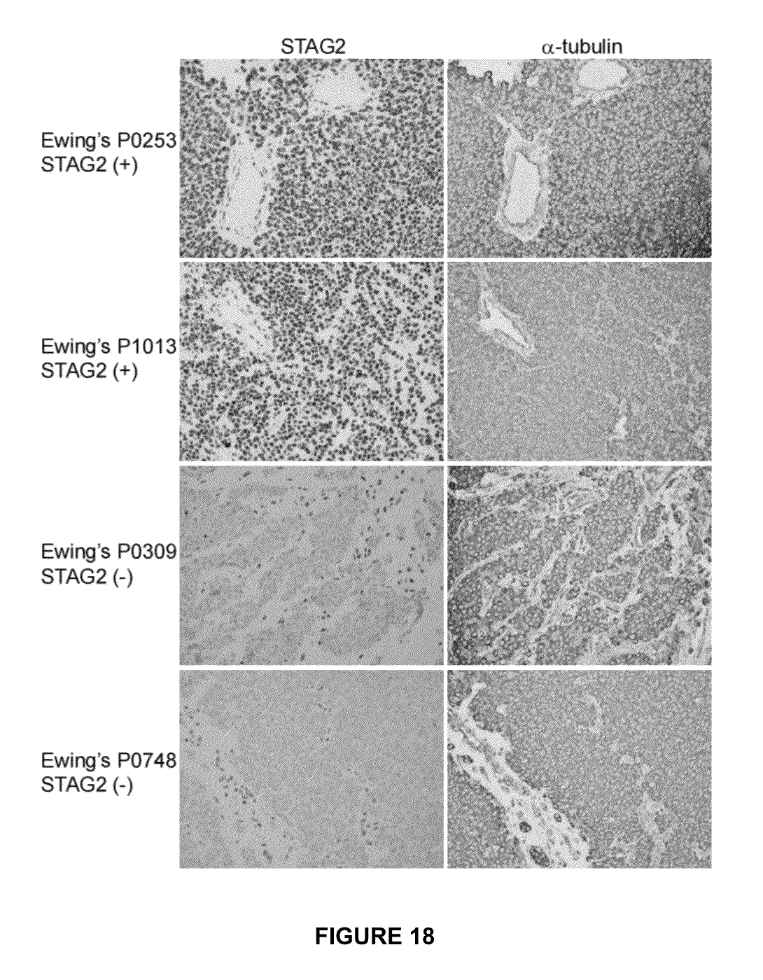

Expression of STAG2 in glioblastoma, melanoma, lymphoma, and Ewing's sarcoma primary human tumor samples was evaluated using the J-12 and DM1A antibodies. Robust STAG2 expression was observed in all non-neoplastic tissues studied (see FIG. 15). In contrast, 19% of glioblastomas, 19% of melanomas, 21% of Ewing's sarcomas, and 2% of lymphomas had completely lost expression of STAG2, with occasional tumors demonstrating intratumoral heterogeneity (see FIGS. 2B-C and 16-18). Taken together, these data indicate that STAG2 is inactivated in a substantial fraction of human cancers.

### Example 5

### Somatic Cell Gene Targeting

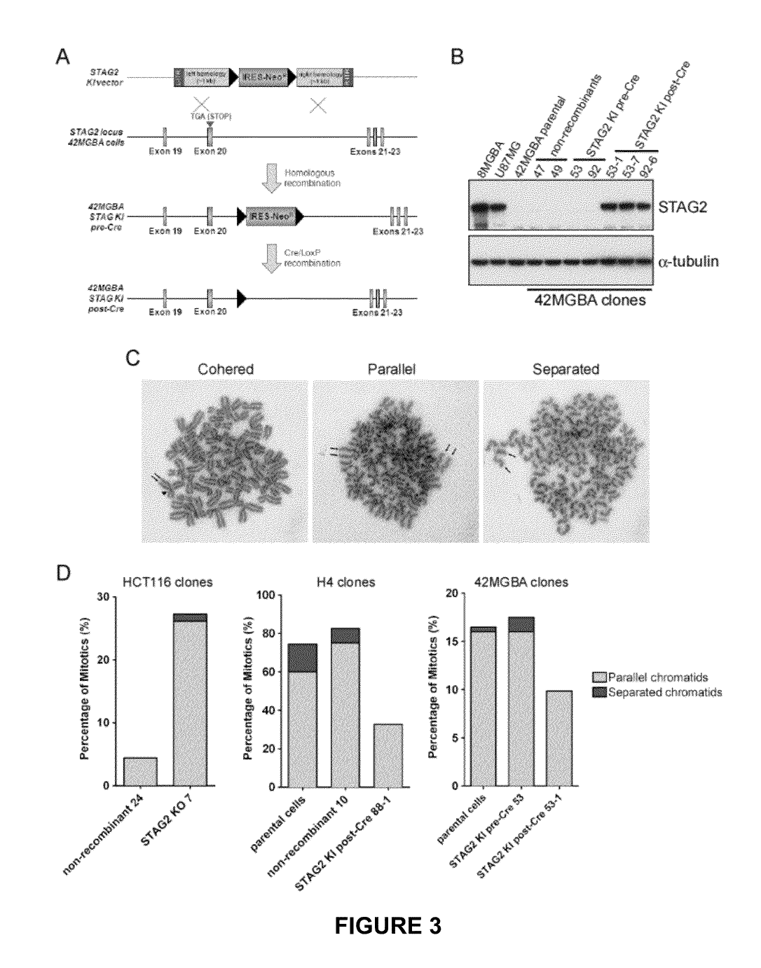

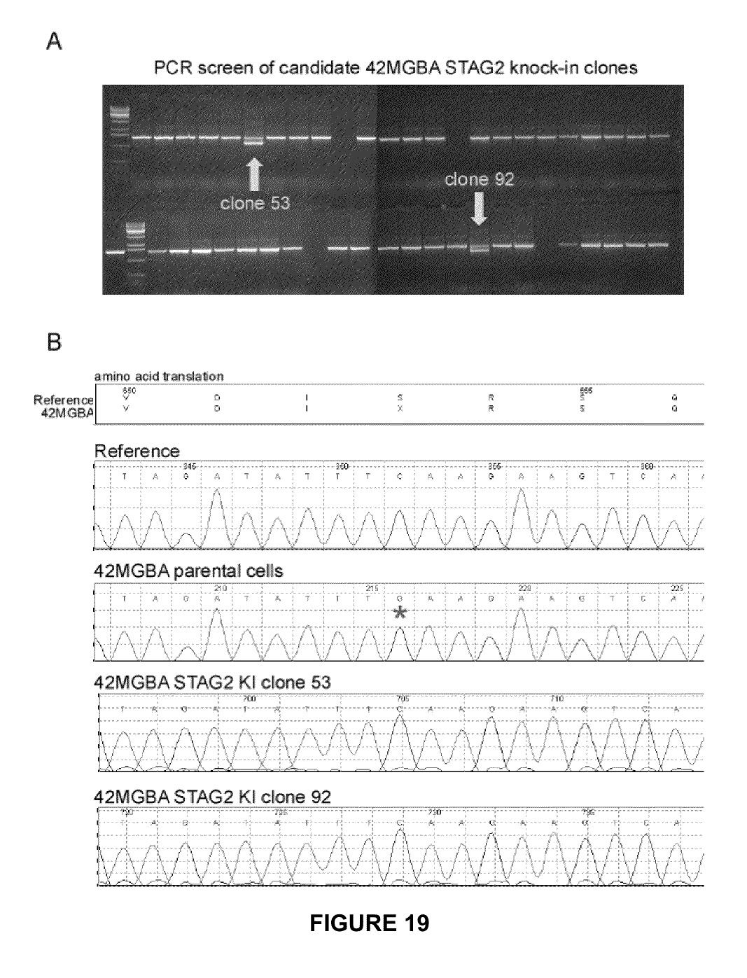

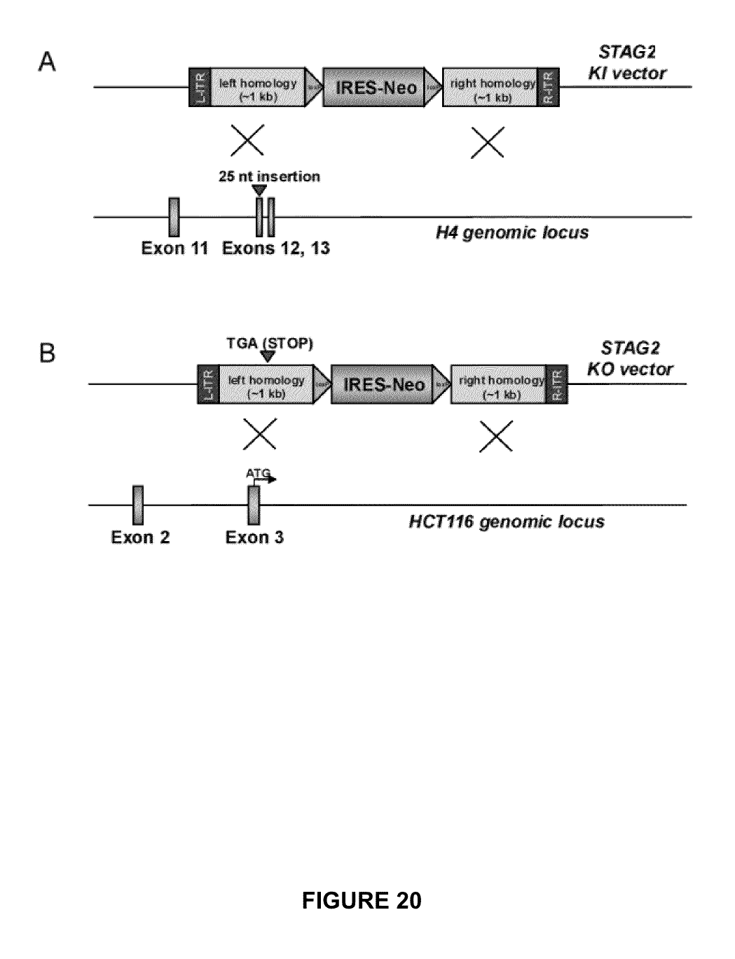

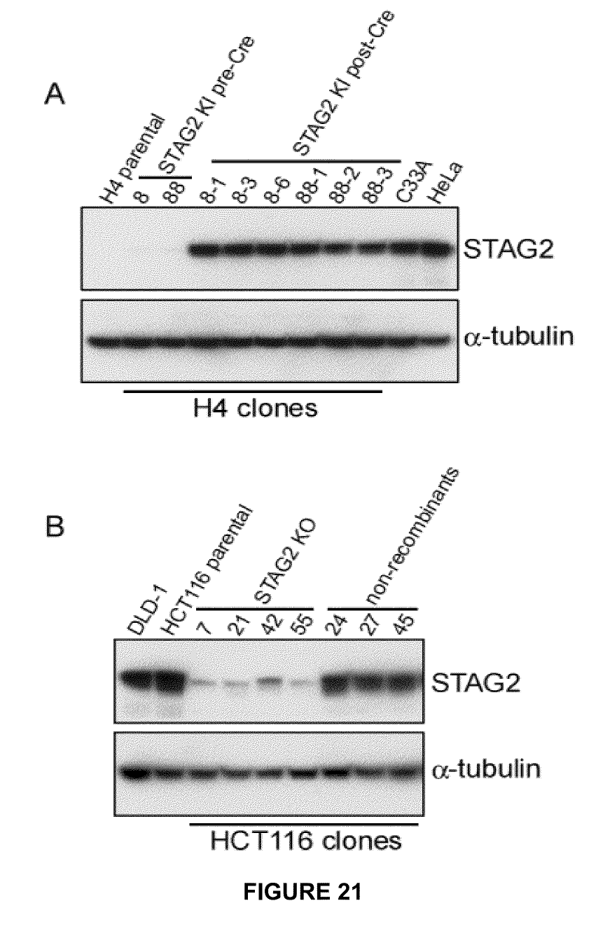

To create experimental systems suitable for determining the functional significance of STAG2 inactivation in cancer pathogenesis, human somatic cell gene targeting was used to correct the endogenous mutations of STAG2 in two aneuploid glioblastoma cell lines. H4 cells are reported to be hypertriploid with modal chromosome number 73, range 63-78, and 42MGBA cells are hypertetraploid with modal chromosome number 89, range 88-95. Adeno-associated virus (AAV) targeting vectors were constructed and used to correct the 25-bp insertion mutation in exon 12 of H4 cells and the nonsense mutation in exon 20 of 42MGBA cells (see FIGS. 3A, 19 and 20). Western blots were then performed to document that correction of the mutations in H4 and 42MGBA knock-in (KI) cells led to re-expression of STAG2 protein (see FIGS. 3B and 21A). Similarly, human somatic cell gene targeting was also used to introduce a nonsense mutation into codon 6 of the endogenous wild-type allele of STAG2 in HCT116, a near-diploid colorectal cancer cell line with stable karyotype (see FIGS. 20B and 21B).

It has been demonstrated that the cohesin complex plays two different roles in eukaryotic cell biology—as a structural complex that holds together sister chromatids following DNA replication to ensure faithful segregation of chromosomes into daughter cells, and also as a regulator of chromatin architecture and transcription. Therefore, STAG2 knock-in (H4 and 42MGBA) and knockout (HCT116) cells were treated with either taxol or nocodazole to induce mitotic arrest, chromatids were visualized by Giemsa staining, and the percentage of parallel or separated chromatids were scored in a blinded fashion (see FIG. 3C-D). STAG2-proficient HCT116 cells demonstrated virtually perfect sister chromatid cohesion that was markedly abrogated upon knockout of STAG2. In contrast, STAG2-deficient H4 and 42MGBA cells demonstrated substantial defects in sister chromatid cohesion that were largely reverted upon targeted correction of STAG2.

### Example 6

### Roles of STAG2

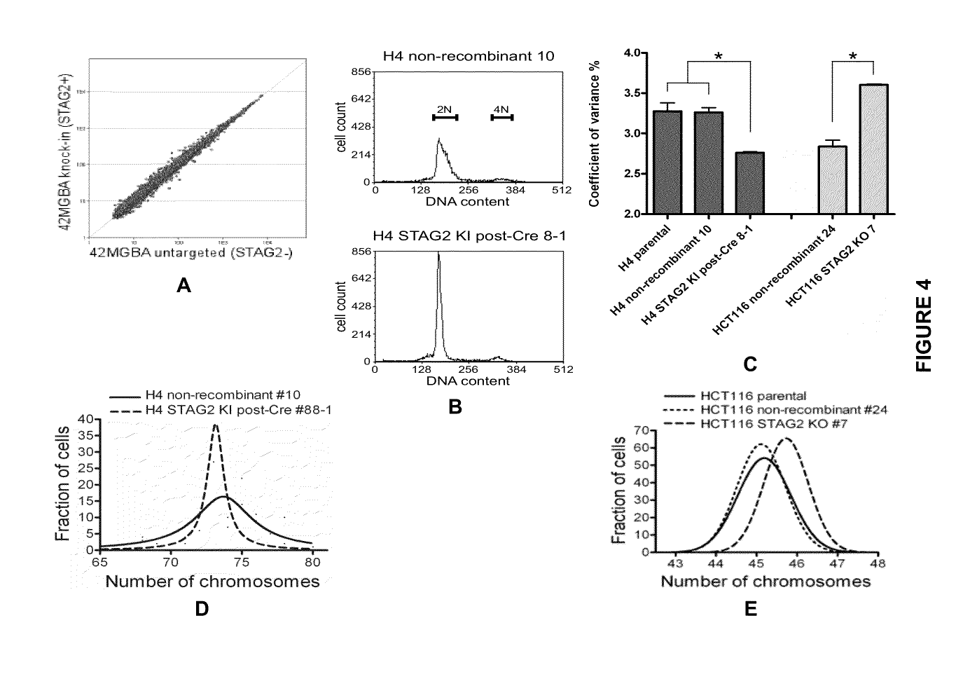

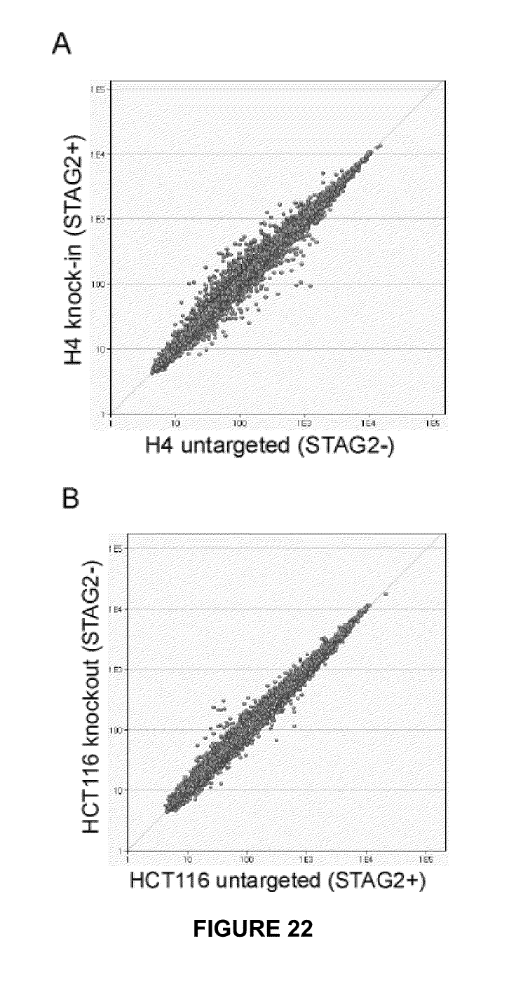

To identify a potential role for STAG2 in regulating transcription in human cancer cells, expression microarrays were used to measure global gene expression profiles in the three different sets of isogenic STAG2-corrected and STAG2 KO cells. As depicted in Tables 3-5 and FIGS. 4A and 22, expression profiles of STAG2-proficient and deficient cells were remarkably similar (i.e. only 16/28,869 genes [0.06%] were modulated >1.5-fold in STAG2-corrected 42MGBA cells), indicating that STAG2 is not likely to play a global role in the regulation of gene expression in human cancer. Furthermore, no genes were recurrently up or down regulated by STAG2 in more than one cell line. For example, Angiopoietin-2 expression was increased 8-fold in multiple clones of STAG2 KO HCT116 cells, but was not correspondingly downregulated in STAG2 KI H4 or 42MGBA cells. Taken together, this expression profiling data suggests that the role of STAG2 in cancer pathogenesis is not due to global transcriptional changes or modulating the expression of specific tumor-promoting or suppressing genes.

TABLE 3
Group 1: H4 parental cells and non-recombinant clones 10 and 12
Group 2: H4 STAG2 knock-in clones 8-1, 8-3, and 88-1
Statistics: t-test
Correction: Benjamini and Hochberg
Data Transformation: Log Transformed

| Ref. | Grp1 Mean | Grp1 SEM | Grp2 Mean | Grp2 SEM | Ratio | Direction | p-value |
|---|---|---|---|---|---|---|---|
| A | 10.2712 | 0.3675 | 6.5456 | 0.3165 | 13.23 | Down | 0.002 |
| B | 9.7974 | 0.2751 | 6.7493 | 0.1257 | 8.27 | Down | 0.001 |
| C | 5.5683 | 0.1801 | 8.2272 | 0.1509 | 6.32 | Up | 0.000 |
| D | 4.8819 | 0.4679 | 7.4870 | 0.2946 | 6.08 | Up | 0.009 |
| E | 4.0628 | 0.1991 | 6.6555 | 0.6868 | 6.03 | Up | 0.022 |
| F | 4.2749 | 0.1639 | 6.7521 | 0.3655 | 5.57 | Up | 0.003 |
| G | 8.6153 | 0.8492 | 6.1471 | 0.1015 | 5.53 | Down | 0.045 |
| H | 7.1050 | 0.3730 | 4.6572 | 0.2281 | 5.46 | Down | 0.005 |
| I | 5.9822 | 0.5267 | 8.4280 | 0.0463 | 5.45 | Up | 0.010 |
| J | 7.6110 | 0.2287 | 5.3149 | 0.1626 | 4.91 | Down | 0.001 |
| K | 6.4001 | 0.5292 | 8.6927 | 0.4043 | 4.90 | Up | 0.026 |
| L | 6.0177 | 0.5928 | 8.2370 | 0.1396 | 4.66 | Up | 0.022 |
| M | 6.8620 | 0.4401 | 9.0668 | 0.1181 | 4.61 | Up | 0.008 |
| N | 6.7176 | 0.1232 | 8.9105 | 0.0206 | 4.57 | Up | 0.000 |
| O | 5.8429 | 0.4912 | 8.0132 | 0.1146 | 4.50 | Up | 0.013 |
| P | 9.8246 | 0.4899 | 7.6652 | 0.2323 | 4.47 | Down | 0.016 |
| Q | 6.7761 | 0.4615 | 8.9092 | 0.1903 | 4.39 | Up | 0.013 |
| R | 5.9754 | 0.3864 | 8.0889 | 0.4034 | 4.33 | Up | 0.019 |
| S | 3.6503 | 0.0985 | 5.6848 | 0.3286 | 4.10 | Up | 0.004 |
| T | 5.7741 | 0.3952 | 7.8069 | 0.3620 | 4.09 | Up | 0.019 |
| U | 6.6259 | 0.2985 | 8.6509 | 0.1496 | 4.07 | Up | 0.004 |
| V | 7.7160 | 0.2965 | 9.7379 | 0.1819 | 4.06 | Up | 0.004 |
| W | 9.3063 | 0.3424 | 7.3361 | 0.4572 | 3.92 | Down | 0.026 |
| X | 5.1012 | 0.4226 | 6.9820 | 0.0511 | 3.68 | Up | 0.012 |
| Y | 5.4503 | 0.5498 | 7.3222 | 0.1496 | 3.66 | Up | 0.030 |
| Z | 7.4863 | 0.3523 | 5.6156 | 0.1405 | 3.66 | Down | 0.008 |
| AA | 8.2359 | 0.3763 | 10.0819 | 0.0660 | 3.60 | Up | 0.008 |
| BB | 10.5931 | 0.3318 | 8.7751 | 0.1800 | 3.53 | Down | 0.009 |
| CC | 9.9060 | 0.3948 | 8.1060 | 0.0619 | 3.48 | Down | 0.011 |
| DD | 7.9164 | 0.3761 | 9.7034 | 0.2032 | 3.45 | Up | 0.014 |
| EE | 5.3140 | 0.3832 | 7.0540 | 0.4864 | 3.34 | Up | 0.048 |
| FF | 8.0258 | 0.1959 | 6.3615 | 0.2293 | 3.17 | Down | 0.005 |
| GG | 7.7192 | 0.0992 | 9.3762 | 0.0779 | 3.15 | Up | 0.000 |
| HH | 4.9307 | 0.3210 | 6.5731 | 0.2368 | 3.12 | Up | 0.015 |
| II | 4.6102 | 0.3729 | 6.2208 | 0.2955 | 3.05 | Up | 0.028 |
| JJ | 7.0668 | 0.5354 | 8.6585 | 0.1891 | 3.01 | Up | 0.049 |

| Ref. | Gene ID | Gene Name | Ontologies |
|---|---|---|---|
| A | MMP3 | Matrix metallopeptidase 3 (stromelysin | proteolysis |
| | | 1, progelatinase) | |
| B | ST8SIA4 | ST8 alpha-N-acetyl-neuraminide alpha- | protein amino acid glycosylation |
| | | 2,8-sialyltransferase 4 | |
| C | FRMD4B | FERM domain containing 4B | |
| D | FAP | Fibroblast activation protein, alpha | biopolymer catabolic process |
| E | TAC1 | Tachykinin, precursor 1 | positive regulation of acute inflammatory |
| | | | response, natriuresis |
| F | DCN | Decorin | organ morphogenesis, peptide cross-linking |
| | | | via chondroitin 4-sulfate glycosaminoglycan |
| G | OR51B4 | Olfactory receptor, family 51, | G-protein coupled receptor protein (GPCRP) |
| | | subfamily B, member 4 | signaling pathway |
| H | RGS18 | Regulator of G-protein signaling 18 | regulation of GPCRP signaling pathway |
| I | DAPK1 | Death-associated protein kinase 1 | protein kinase, anti-apoptosis |
| J | CYB5A | Cytochrome b5 type A (microsomal) | electron transport chain |
| K | AMTN | Amelotin | cell adhesion, biomineral formation |
| L | GABRQ | GABA receptor, theta | ion transport |
| M | LEPREL1 | Leprecan-like 1 | oxidation reduction |
| N | STAG2 | Stromal antigen 2 | cell cycle, chromosome segregation |
| O | ANK3 | Ankyrin 3, node of Ranvier (ankyrin G) | signal transduction, establishment of protein |
| | | | localization |
| P | ADAMTS19 | ADAM metallopeptidase with | proteolysis |
| | | thrombospondin type 1 motif, 19 | |
| Q | MGP | Matrix Gla protein | cartilage condensation |
| R | SRGN | Serglycin | negative regulation of bone mineralization |
| S | — | | |
| T | MME | Membrane metallo-endopeptidase | proteolysis |
| U | LPAR1 | Lysophosphatidic acid receptor 1 | GPCRP signaling pathway |
| V | DNER | Delta/notch-like EGF repeat containing | neuron migration |
| W | PTGS2 | Prostaglandin-endoperoxide synthase 2 | fatty acid biosynthetic process |
| | | (prostaglandin G/H synthase) | |
| X | PREX2 | Phosphatidylinositol-3,4,5- | GPCRP signaling pathway |
| | | trisphosphate-dependent Rac exchange | |
| | | factor 2 | |
| Y | EPB41L3 | Erythrocyte membrane protein band | cortical actin cytoskeleton organization |
| | | 4.1-like 3 | |
| Z | CDH7 | Cadherin 7, type 2 | homophilic cell adhesion |
| AA | CD24 | CD24 molecule | response to hypoxia |
| BB | TXNIP | Thioredoxin interacting protein | response to oxidative stress |
| CC | SEMA3D | Semaphorin 3D | nervous system development |
| DD | ALDH1A3 | Aldehyde dehydrogenase 1 family, | kidney development, optic cup morphogenesis |
| | | member A3 | |
| EE | — | GABA A receptor, epsilon | ion transport, GABA signaling pathway |
| FF | AOAH | Acyloxyacyl hydrolase (neutrophil) | lipid metabolic process, inflammatory rspnse |
| GG | TNFRSF21 | Tumor necrosis factor receptor | apoptosis, signal transduction |
| | | superfamily, member 21 | |
| HH | SCN3A | Sodium channel, voltage-gated, type | sodium ion transport |
| | | III, alpha subunit | |
| II | C7orf69 | Chromosome 7 open reading frame 69 | |
| JJ | TOX | Thymocyte selection-associated high | DNA binding |
| | | mobility group box | |

TABLE 4
Group 1: 42MGBA parental cells and pre-Cre clones 53 and 92
Group 2: 42MGBA STAG2 knock-in clones 53-1, 53-7, and 92-6
Statistics: t-test
Correction: Benjamini and Hochberg
Data Transformation: Log Transformed

| Ref. | Grp1 Mean | Grp1 SEM | Grp2 Mean | Grp2 SEM | Ratio | Direction | p-value |
|---|---|---|---|---|---|---|---|
| A | 6.15880 | 0.3866 | 7.6770 | 0.2608 | 2.86 | Up | 0.031 |
| B | 4.2746 | 0.3004 | 5.3110 | 0.1912 | 2.05 | Up | 0.044 |
| C | 4.5495 | 0.1372 | 5.4141 | 0.2706 | 1.82 | Up | 0.046 |
| D | 7.7872 | 0.2043 | 6.9765 | 0.0411 | 1.75 | Down | 0.018 |
| E | 4.4900 | 0.2242 | 5.2741 | 0.0829 | 1.72 | Up | 0.030 |
| F | 6.2648 | 0.2173 | 7.0278 | 0.1582 | 1.70 | Up | 0.047 |
| G | 6.2226 | 0.1259 | 5.4879 | 0.0834 | 1.66 | Down | 0.008 |
| H | 6.6446 | 0.0747 | 5.9433 | 0.2342 | 1.63 | Down | 0.046 |
| I | 4.7247 | 0.2282 | 4.0496 | 0.0467 | 1.60 | Down | 0.044 |
| J | 4.8573 | 0.0934 | 5.5255 | 0.2091 | 1.59 | Up | 0.043 |
| K | 5.6906 | 0.1293 | 6.3526 | 0.0752 | 1.58 | Up | 0.011 |
| L | 4.5136 | 0.0734 | 5.1756 | 0.0836 | 1.58 | Up | 0.004 |
| M | 5.7196 | 0.0943 | 6.3651 | 0.1795 | 1.56 | Up | 0.033 |
| N | 7.2171 | 0.0293 | 7.8291 | 0.0759 | 1.53 | Up | 0.002 |
| O | 5.9287 | 0.1098 | 5.3120 | 0.0929 | 1.53 | Down | 0.013 |
| P | 7.6471 | 0.0472 | 7.0578 | 0.1564 | 1.50 | Down | 0.023 |

| Ref. | Gene ID | Gene Name | Ontologies |
|---|---|---|---|
| A | TGA6 | Integrin, alpha 6 | cell-matrix adhesion, signal transduction |
| B | KITLG | KIT ligand | hematopoiesis, cell survival and proliferation |
| C | PLCB4 | Phospholipase C, beta 4 | lipid catabolic process, signal transduction |
| D | BDKRB1 | Bradykinin receptor B1 | G-protein coupled receptor, |
| | | | regulation of inflammation and vascular tone |
| E | CARD16 | Caspase recruitment domain family, | proteolysis, regulation of apoptosis |
| | | member 16 | |
| F | EPS8 | Epidermal growth factor receptor | signal transduction, cell proliferation |
| | | pathway substrate 8 | |
| G | ncrna: | ncrna: ENSG00000210467 | |
| H | TSHZ1 | Teashirt zinc finger homeobox 1 | regulation of transcription, |
| | | | developmental pattern formation |
| I | NT5E | 5-nucleotidase, ecto (CD73) | purine nucleotide biosynthetic process |
| J | — | Chr 6 ORF 155 | |
| K | UQCRFS1 | Ubiquinol-cytochrome c reductase, | cellular metabolism, electron transport chain |
| | | Rieske iron-sulfur polypeptide 1 | |
| L | — | — | |
| M | ELMO1 | Engulfment and cell motility 1 | cell motility, phagocytosis, apoptosis |
| N | ALDH1L2 | Aldehyde dehydrogenase 1 family, | metabolic process |
| | | member L2 | |
| O | — | 5ncrna: ENSG00000199568 | |
| P | FBLN5 | Fibulin 5 | cell-matrix adhesion |

TABLE 5
Group 1: HCT116 STAG2 non-recombinant clones 24 and 27
Group 2: HCT116 STAG2 knockout clones 7 and 21
Statistics: t-test
Correction: Benjamini and Hochberg
Data Transformation: Log Transformed

| Ref. | Grp1 Mean | Grp1 SEM | Grp2 Mean | Grp2 SEM | Ratio | Direction | p-value |
|---|---|---|---|---|---|---|---|
| A | 4.8069 | 0.1443 | 7.8713 | 0.0645 | 8.37 | Up | 0.003 |
| B | 5.2230 | 0.1343 | 7.7417 | 0.0497 | 5.73 | Up | 0.003 |
| C | 9.2903 | 0.0076 | 6.8549 | 0.2890 | 5.41 | Down | 0.014 |
| D | 4.6207 | 0.0605 | 6.7285 | 0.1329 | 4.31 | Up | 0.005 |
| E | 5.1775 | 0.1844 | 6.8892 | 0.1114 | 3.28 | Up | 0.015 |
| F | 5.2243 | 0.1772 | 3.7052 | 0.1321 | 2.87 | Down | 0.021 |
| G | 7.7983 | 0.0275 | 9.0658 | 0.1807 | 2.41 | Up | 0.020 |
| H | 6.3049 | 0.0087 | 7.4512 | 0.0886 | 2.21 | Up | 0.006 |
| I | 4.6207 | 0.1529 | 3.4761 | 0.0503 | 2.21 | Down | 0.019 |
| J | 3.8346 | 0.1619 | 4.9552 | 0.1310 | 2.17 | Up | 0.033 |
| K | 6.4773 | 0.1765 | 7.5855 | 0.1829 | 2.16 | Up | 0.049 |
| L | 7.1029 | 0.0904 | 8.1371 | 0.0864 | 2.05 | Up | 0.014 |
| M | 4.1376 | 0.0279 | 5.1599 | 0.1105 | 2.03 | Up | 0.012 |
| N | 6.9081 | 0.2230 | 5.8948 | 0.0487 | 2.02 | Down | 0.047 |
| O | 6.3336 | 0.0267 | 7.3363 | 0.1681 | 2.00 | Up | 0.028 |

| Ref. | Gene ID | Gene Name | Ontologies |
|---|---|---|---|
| A | ANGPT2 | Angiopoietin 2 | signal transduction, |
| | | | negative regulator of angiogenesis |
| B | ITGB8 | Integrin, beta 8 | ganglioside metabolic process, cell-matrix adhesion |
| C | STAG2 | Stromal antigen 2 | cell cycle, chromosome segregation |
| D | TM4SF18 | Transmembrane 4 L six family | membrane function |
| | | member 18 | |
| E | SCML1 | Sex comb on midleg-like 1 | anatomical structure morphogenesis, |
| | | | transcriptional regulation |
| F | — | ncrna: ENSG00000200662 | |
| G | EGR1 | Early growth response 1 | negative regulation of transcription from RNA |
| | | | polymerase II promoter |
| H | SLCO3A1 | Solute carrier organic anion | ion transport |
| | | transporter family, member 3A1 | |
| I | — | ncrna: ENSG00000207356 | |
| J | SLC40A1 | Solute carrier family 40, member 1 | iron transport and homeostasis |
| K | C4orf34 | Chr 4 ORF 34 | protein binding, membrane component |
| L | TP53INP1 | Tumor protein p53 inducible | induction of apoptosis, cell cycle arrest |
| | | nuclear protein 1 | |
| M | RASGRP1 | RAS guanyl releasing protein 1 | Ras protein signal transduction, cell differentiation |
| N | — | Zinc finger protein 487 | |
| O | DAPK1 | Death-associated protein kinase 1 | protein kinase, anti-apoptosis |

The cell-cycle profiles of the three isogenic sets of STAG2 proficient and deficient cells were also evaluated. STAG2-proficient and deficient cells had similar percentages of cells in both G1 (2N) and G2/M (4N) (see FIG. 4B). Strikingly, however, the width of the 2N and 4N peaks was substantially higher in each of the STAG2-deficient cell lines than in their otherwise isogenic STAG2-proficient counterparts (see FIG. 4B). The coefficient of variance (CV) is a quantitative measure of this width, reflecting the fraction of cells in a 2N or 4N population with a chromosome count different from that of the modal number. The CV of STAG2-deficient cells was substantially larger than STAG2-proficient cells, which is consistent with STAG2 inactivation causing altered chromosome counts (i.e. aneuploidy) in these cancer cells (see FIG. 4C).

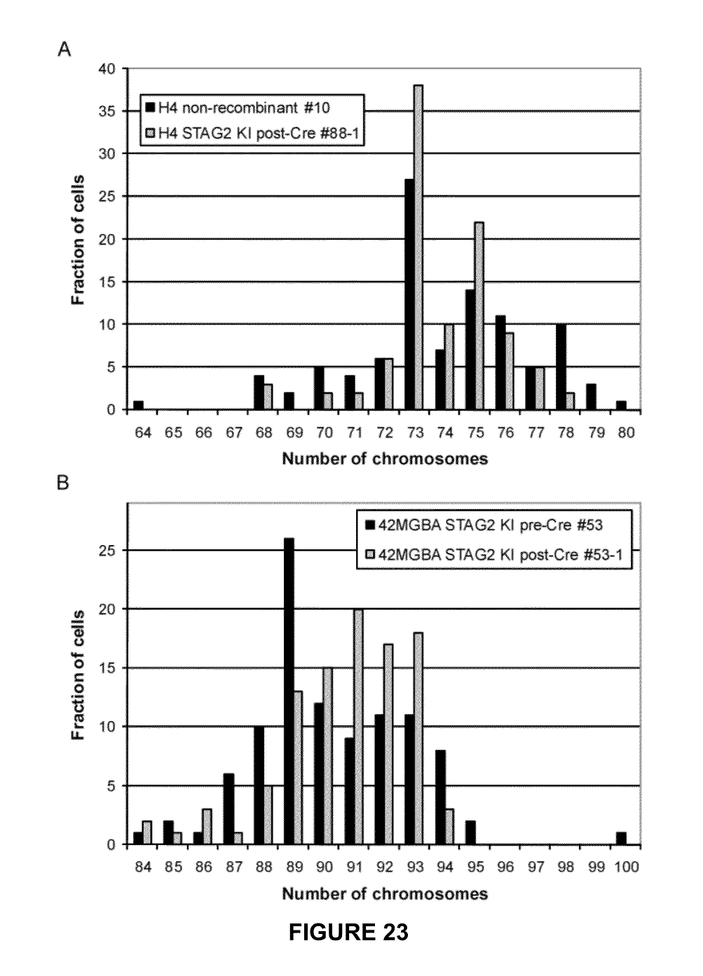

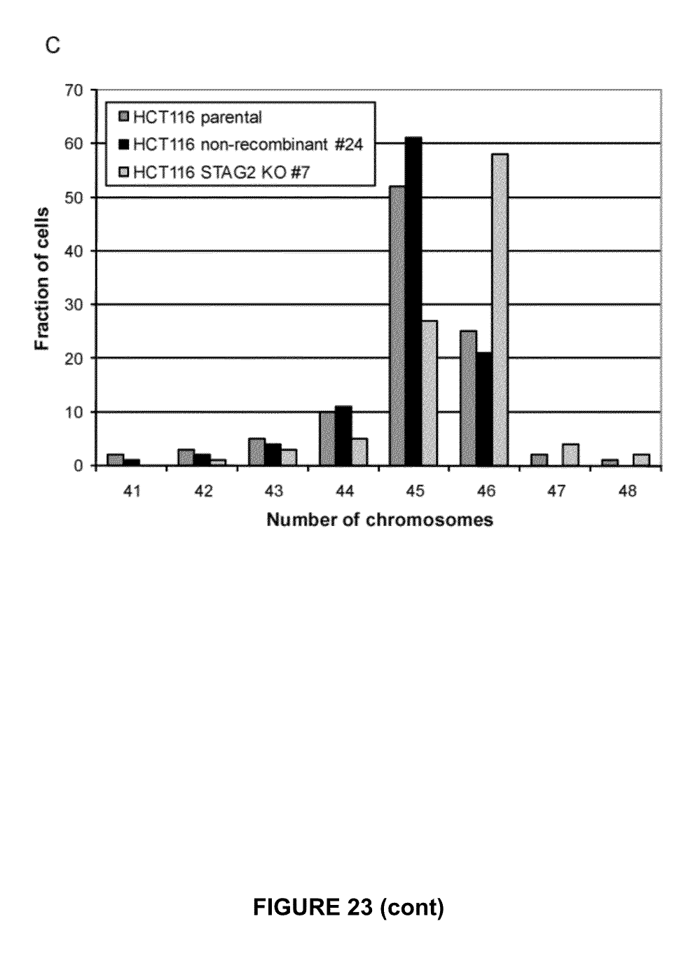

Karyotypic analysis of these isogenic sets of cells was also performed. To do this, metaphase arrested cells were stained with Wright's reagent and chromosome counts performed in a blinded fashion. As depicted in FIGS. 4D and 23A-B, H4 and 42MGBA STAG2 mutant cells had a wider distribution of chromosome counts than their STAG-corrected KI derivatives. Similarly, HCT116 STAG2-proficient cells had a modal chromosome count of 45, whereas their STAG2-deleted derivatives had a modal chromosome count of 46 and a wider range of chromosome counts, which is consistent with the observations in the preceding paragraph (see FIGS. 4E and 23C).

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1. An isolated polynucleotide encoding a STAG2 polypeptide associated with at least one chromosomal aberration.

2. The isolated polynucleotide of claim 1, wherein the at least one chromosomal aberration is aneuploidy.

3. The isolated polynucleotide of any one of claim 1 or 2, wherein the polynucleotide comprises a sequence corresponding to positions 405 to 4211 of SEQ ID NO: 1 and further comprises at least one insertion, deletion or substitution within the sequence as described in Table 1.

4. An isolated polynucleotide that is capable of detecting a STAG2 polynucleotide associated with at least one chromosomal aberration by specifically hybridizing to the STAG2 polynucleotide or its complement under hybridization and wash conditions selected from the group consisting of:
a) 7% sodium dodecyl sulfate (SDS), 0.5 M NaPO₄, 1 mM EDTA at 50° C. with washing in 2×SSC, 0.1% SDS at 50° C.;
b) 7% sodium dodecyl sulfate (SDS), 0.5 M NaPO₄, 1 mM EDTA at 50° C. with washing in 1×SSC, 0.1% SDS at 50° C.;
c) 7% sodium dodecyl sulfate (SDS), 0.5 M NaPO₄, 1 mM EDTA at 50° C. with washing in 0.5×SSC, 0.1% SDS at 50° C.;
d) 7% sodium dodecyl sulfate (SDS), 0.5 M NaPO₄, 1 mM EDTA at 50° C. with washing in 0.1×SSC, 0.1% SDS at 50° C.; and
e) 7% sodium dodecyl sulfate (SDS), 0.5 M NaPO₄, 1 mM EDTA at 50° C. with washing in 0.1×SSC, 0.1% SDS at 65° C.

5.-47. (canceled)

48. An isolated STAG2 polypeptide encoded by the isolated polynucleotide of claim 1.

49. An isolated STAG2 polypeptide encoded by the isolated polynucleotide of claim 2.

50. An isolated STAG2 polypeptide encoded by the isolated polynucleotide of claim 3.

51. A method of determining a clinical course of treatment for a subject with at least one tumor comprising:
(a) in a sample of the tumor obtained from the subject, detecting the presence or absence of a STAG2 polynucleotide or polypeptide associated with at least one chromosomal aberration in a tumor sample obtained from the subject;
(b) correlating the presence or absence of the STAG2 polynucleotide or polypeptide with the clinical course of treatment.

52. The method of claim 51, wherein the at least one tumor is obtained from a cancer or tumor selected from the group consisting of bladder cancer, glioblastoma multiforme, Ewing's sarcoma, melanoma, and immunoblastic lymphoma.

53. The method of claim 51, wherein detecting the presence or absence of the STAG2 polynucleotide or polypeptide comprises contacting the sample with an antibody.

54. The method of claim 53, wherein the antibody is a JS-12 antibody.

55. The method of claim 51, wherein detecting the presence or absence of the STAG2 polynucleotide or polypeptide comprises determining a relative level of expression of the STAG2 polynucleotide or polypeptide compared to a control and correlating the relative level of expression with the clinical course of treatment.

56. The method of claim 55, wherein detecting the presence or absence of the STAG2 polynucleotide or polypeptide comprises contacting the sample with an antibody.

57. The method of claim 51, wherein correlating the presence or absence of the STAG2 polynucleotide or polypeptide with the clinical course of treatment comprises determining the likelihood that the tumor will metastasize.

58. The method of claim 51, wherein correlating the presence or absence of the STAG2 polynucleotide or polypeptide with the clinical course of treatment comprises determining the likelihood that the tumor will respond to the administration of at least one therapeutic agent.

59. The method of claim 51, wherein the STAG2 polynucleotide is determined to comprise at least one nucleotide insertion, nucleotide deletion, or substitution in the STAG2 gene set forth in Table 1.
